(19) United States
(12) Patent Application Publication
(10) Pub. No.: US 2023/0082584 A1
(43) Pub. Date: Mar. 16, 2023

(54) COMPOSITIONS AND METHODS FOR IDENTIFYING HOST CELL TARGET PROTEINS FOR TREATING RNA VIRUS INFECTIONS

(71) Applicant: The Regents of the University of California, Oakland, CA (US)

(72) Inventors: Jennifer A. Doudna, Berkeley, CA (US); Chun-Hao Huang, San Francisco, CA (US); Ko-Chuan Lee, San Francisco, CA (US)

(21) Appl. No.: 17/800,417

(22) PCT Filed: Mar. 29, 2021
(86) PCT No.: PCT/US21/24657

§ 371 Date: Aug. 17, 2022

Related U.S. Application Data
(60) Provisional application No. 63/002,576, filed on Mar. 31, 2020.

(51) Int. Cl. C12N 15/10

(52) U.S. Cl. CPC C12N 15/1055; C12N 15/1086

(57) ABSTRACT

The present disclosure provides methods for testing whether a target protein regulates viral RNA translation. The methods comprise a) introducing into a test host cell (where the test host cell comprises a catalytically inactive or a catalytically active CRISPR/Cas effector polypeptide): i) a reporter nucleic acid comprising a nucleotide sequence encoding a bicistronic translation monitor; and ii) a regulatory nucleic acid comprising a nucleotide sequence encoding a single guide RNA (sgRNA) that comprises a targeting sequence that specifically binds to a target sequence within a nucleic acid encoding the target protein; and b) detecting expression of the reporter proteins to determine whether a target protein regulates viral RNA translation via a Cap-dependent element or a Cap-independent element based on the expression of the reporter proteins in the test host cell as compared to the expression in the control host cell. Kits for conducting the methods disclosed herein are also provided.

### CROSS-REFERENCE

This application claims the benefit of U.S. Provisional Patent Application No. 63/002,576, filed Mar. 31, 2020, which application is incorporated herein by reference in its entirety.

### INTRODUCTION

Viral infection is a multistep process involving complex interplay between viral life cycle and host immunity One defense mechanism that hosts use to protect cells against the virus are nucleic-acid-mediated surveillance systems, such as RNA interference-driven gene silencing and CRISPR-Cas mediated gene editing. Understanding these virus-host interactions has opened up the prospect of modulating expression of specific genes and has led to the emergence of powerful tools for studying gene functions or serving as potential therapeutic agents. Another important stage for host cells to combat virus replication is translational control—a competition between viral and host mRNAs for translational machinery.

Translational regulation is particularly important for the life cycle of RNA viruses. Hepatitis C virus (HCV), which infects 170 million people worldwide and can lead to liver cancer, uses an internal ribosome entry site (IRES) to facilitate efficient translation of viral mRNA by directly recruiting host ribosomal subunits and translation initiation factors in a cap-independent manner Other RNA viruses, such as Coronavirus, employ cap-dependent translation mechanisms or ribosomal frameshifting to perform protein synthesis. While efforts to characterize structural features of viral RNA have led to a better understanding of translational regulation, no systematical approaches to identify important host genes for controlling viral translation have been developed and little is known about how to regulate the host-virus translational interaction to prevent and treat infections caused by RNA viruses.

There is a need in the art for methods of identifying therapeutic drug targets to treat RNA virus infections.

### SUMMARY

The present disclosure provides methods for testing whether a target protein (e.g., a host cell target protein) regulates viral RNA translation. The methods comprise: a) introducing into a test host cell comprising a catalytically inactive or a catalytically active CRISPR/Cas effector polypeptide: i) a reporter nucleic acid comprising a nucleotide sequence encoding a bicistronic translation monitor; and a ii) regulatory nucleic acid comprising a nucleotide sequence encoding a single guide RNA (sgRNA) that comprises a targeting sequence that specifically binds to a target sequence within a nucleic acid encoding the target protein; and b) detecting expression of the reporter proteins to determine whether a target protein regulates translation via a Cap-dependent element or a Cap-independent element based on the expression of the reporter proteins in the test host cell as compared to the expression in the control host cell. The present disclosure provides kits for performing such methods and comprise: a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and b) a regulatory nucleic acid comprising a nucleotide sequence encoding a single guide RNA (sgRNA) that comprises a targeting sequence that specifically binds to a target sequence within a nucleic acid encoding the target protein.

The compositions and methods disclosed herein can be used to identify new therapeutics targets or repurposed drug targets for blocking viral RNA translation. The compositions and methods can also be used to identify important domains within target proteins that are required for regulating (viral RNA translation) and can inform drug design and development for treating RNA viruses.

### BRIEF DESCRIPTION OF THE DRAWINGS

FIG. 1. A high-throughput system to identify host genes required for translation initiation of viral RNA. A diagram of the bicistronic reporter system in combination with CRISPR-dCas9. RFP is translated by a cap-dependent mechanism, and GFP expression is dependent on a functional viral RNA. The human liver cancer HepG2 cells expressing dCas9-KRAB (or dCas9-VPR) and the bicistronic reporter are then transduced with BFP-linked sgRNAs targeting components required for translation of viral RNA.

FIG. 2A-2B. Validation of the combinatorial bicistronic reporter and CRISPR-dCas9 system. (A) The representative fluorescent images and flow cytometry analysis for HCV IRES RNA are shown. RFP and GFP signals indicate cap-dependent and cap-independent translation activities, respectively. BFP indicates sgRNA expression. (B) The representative fluorescent images for other viral (EMCV) and cellular (MYC and XIAP) IRES RNAs are shown. RFP and GFP signals indicate cap-dependent and cap-independent translation activities, respectively.

FIG. 3. Validation of the bicistronic reporter system in multiple human cell lines. Flow cytometry analysis of the 7 human liver cancer cell lines stably expressing the bicistronic reporter. RFP is translated by a cap-dependent mechanism, and GFP expression is dependent on a functional viral RNA. Parental HepG2 cell lines without the bicistronic reporter was used as a negative control.

FIG. 4. Validation of the CRISPR-dCas9 system in the human liver cancer HepG2 cells expressing the bicistronic reporter. RFP is translated by a cap-dependent mechanism, and GFP expression is dependent on a functional viral RNA. Both RFP and GFP signals were not changed upon the transduction of control non-targeting sgRNA.

FIG. 5. A list of 23 translation- or IRES-related host target candidate genes which were tested by two focused CRISPRi sgRNA libraries.

FIG. 6A-6C. Focused CRISPRi screen identifies genes required for translational regulation of HCV IRES RNA. (A) Quantitative PCR (qPCR) analysis of gene expression in cells upon CRISPRi transduction. (B) Flow cytometry analysis of the change of GFP^{high} cells percentage in cells upon CRISPRi transduction. (C) Flow cytometry analysis of the change of RFP^{high} cells percentage in cells upon CRISPRi transduction.

FIG. 7A-7C. Focused CRISPRi screen identifies some EIF genes required for translational regulation of HCV IRES RNA. (A) Quantitative PCR (qPCR) analysis of gene expression in cells upon CRISPRi transduction. (B) Flow cytometry analysis of the change of GFP^{high} cells percentage in cells upon CRISPRi transduction. (C) Flow cytometry analysis of the change of RFP^{high} cells percentage in cells upon CRISPRi transduction.

FIG. 8. A high-throughput system to identify functional domains of host genes required for translation initiation of viral RNA. A diagram of the bicistronic reporter system in combination with CRISPR-Cas9. RFP is translated by a cap-dependent mechanism, and GFP expression is dependent on a functional viral RNA. The human liver cancer HepG2 cells expressing Cas9 and the bicistronic reporter are then transduced with mCherry-linked sgRNAs targeting sequences in domains of host genes required for translation of viral RNA.

FIG. 9. Validation of the CRISPR-Cas9 system in the human cells expressing the bicistronic reporter. T7 endonuclease cleaves double-stranded DNA at positions of mismatches, enabling detection of Cas9-induced breaks in HEK293T cells due to mutations generated during NHEJ repair. Protospacers were designed to target specific domains of EIF3A, EIF4B, EIF4E and EIF5A. Samples with efficient cleavage contain sub-bands that are smaller than uncleaved PCR product. HEK293T Cells infected with empty vectors or vectors with random sequences were used as negative controls.

FIG. 10-10B. Domain-focused CRISPR strategy was performed with sgRNAs targeting EIF3A, EIF4B, EIF4E and EIF5A in Cas9-expressing HepG2 liver cancer cells. The relative location of each sgRNA to the EIF3A, EIF4B, EIF4E and EIF5A proteins is indicated along the x-axis. Empty vector or non-targeting sgRNAs were used as negative controls. y-axis: Flow cytometry analysis of the change of (A) GFP^{high} cells or (B) RFP^{high} cells percentage in cells upon sgRNA transduction.

FIG. 11. Validation of HCV IRES activity using a monocistronic reporter upon the suppression of EIF genes. Left panel: Flow cytometry analysis of the change of GFP^{high} cells percentage in HEK293 cells upon CRISPRi transfection. The hairpin-assisted monocistronic vector (left panel) is used to validate the result from the bicistronic reporter vector (right panel).

FIG. 12. Validation of HCV IRES activity using orthogonal approaches upon the suppression of EIF genes. Left panel: Flow cytometry analysis of the change of GFP^{high} cells percentage in HepG2 liver cancer cells upon CRISPRi and other viral (EMCV) or cellular (XIAP and MYC) IRES transduction. The result of HCV IRES (from FIG. 11) was used as a comparison. Right panel: Flow cytometry analysis of the change of GFP^{high} cells percentage in HepG2 liver cancer cells upon CRISPRi and HCV IRES transduction. The promoter-deleted vector is used to examine the cryptic promoter activity of HCV IRES.

FIG. 13A-13W provide amino acid sequences of exemplary target proteins in humans. Homologs of these proteins from other organisms can also be used.

FIG. 14A-14P provide amino acid sequences of CRISPR/Cas effector polypeptides.

FIG. 15 provides the nucleotide sequences for HCV IRES.

FIG. 16A-16B provides sgRNA sequences for exemplary target proteins.

FIG. 17 provides exemplary sgRNA sequences for targeting EIF3A.

FIG. 18 provides exemplary sgRNA sequences for targeting EIF4B.

FIG. 19 provides exemplary sgRNA sequences for targeting EIF4E.

FIG. 20 provides exemplary sgRNA sequences for targeting EIF5A.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a modified CRISPR/Cas effector polypeptide/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{D}) of less than 10⁻⁻⁶ M, less than 10⁻⁻⁷ M, less than 10⁻⁸ M, less than 10⁻⁹ M, less than 10⁻¹⁰ M, less than 10 ⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

By “binding domain” it is meant a protein domain that is able to bind non-covalently to another molecule. A binding domain can bind to, for example, a DNA molecule (a DNA-binding domain), an RNA molecule (an RNA-binding domain) and/or a protein molecule (a protein-binding domain) In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

A DNA sequence that “encodes” a particular RNA is a DNA nucleotide sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleotide sequence that is transcribed into mRNA (in the case of DNA) and is translated (in the case of mRNA) into a polypeptide in vitro or in vivo when placed under the control of appropriate regulatory sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence and/or regulate translation of an encoded polypeptide.

As used herein, a “promoter” or a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive expression by the various vectors of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism that can be isolated from a source in nature is naturally occurring.

The term “fusion” as used herein as applied to a nucleic acid or polypeptide refers to two components that are defined by structures derived from different sources. For example, where “fusion” is used in the context of a fusion polypeptide (e.g., a fusion polypeptide comprising a CRISPR/Cas effector polypeptide and a fusion partner(s)), the fusion polypeptide includes amino acid sequences that are derived from different polypeptides. A fusion polypeptide may comprise either modified or naturally-occurring polypeptide sequences (e.g., a first amino acid sequence from a CRISPR/Cas effector polypeptide; and a second amino acid sequence from a protein other than a CRISPR/Cas effector polypeptide, etc.).

The term “fusion polypeptide” refers to a polypeptide which is made by the combination (i.e., “fusion”) of two otherwise separated segments of amino acid sequence, usually through human intervention.

“Heterologous,” as used herein, means a nucleotide or polypeptide sequence that is not found in the native nucleic acid or protein, respectively. For example, in some cases, in a modified CRISPR/Cas effector polypeptide of the present disclosure, a portion of a naturally-occurring CRISPR/Cas effector polypeptide (or a variant thereof) may be fused to a heterologous polypeptide (i.e. an amino acid sequence from a protein other than a CRISPR/Cas effector polypeptide; or an amino acid sequence from another organism). As another example, a modified CRISPR/Cas effector polypeptide of the present disclosure comprises a portion of a naturally-occurring CRISPR/Cas effector (or variant thereof) fused to a heterologous polypeptide, i.e., a polypeptide from a protein other than CRISPR/Cas effector, or a polypeptide from another organism. The heterologous polypeptide may exhibit an activity (e.g., enzymatic activity) that will also be exhibited by the modified CRISPR/Cas effector polypeptide.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. An example of such a case is a DNA (a recombinant) encoding a wild-type protein where the DNA sequence is codon optimized for expression of the protein in a cell (e.g., a eukaryotic cell) in which the protein is not naturally found (e.g., expression of a modified CRISPR/Cas effector polypeptide of the present disclosure in a eukaryotic cell). A codon-optimized DNA can therefore be recombinant and non-naturally occurring while the protein encoded by the DNA may have a wild type amino acid sequence.

Thus, the term “recombinant” polypeptide does not necessarily refer to a polypeptide whose amino acid sequence does not naturally occur. Instead, a “recombinant” polypeptide is encoded by a recombinant non-naturally occurring DNA sequence, but the amino acid sequence of the polypeptide can be naturally occurring (“wild type”) or non-naturally occurring (e.g., a variant, a mutant, etc.). Thus, a “recombinant” polypeptide is the result of human intervention, but may have a naturally occurring amino acid sequence.

A “vector” or “expression vector” is a replicon, such as plasmid, phage, virus, artificial chromosome, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner For instance, a promoter is operably linked to a coding sequence (or the coding sequence can also be said to be operably linked to the promoter) if the promoter affects its transcription or expression.

“Operably linked” refers to a juxtaposition wherein the components so described are in a relationship permitting them to function in their intended manner For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell or a cell line cultured as a unicellular entity or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The terms “recombinant expression vector,” or “DNA construct” are used interchangeably herein to refer to a DNA molecule comprising a vector and an insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified” or “transformed” or “transfected” by exogenous DNA or exogenous RNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change is generally achieved by introduction of new DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: 50169-409X(12)00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “target nucleic acid” as used herein is a polynucleotide (e.g., DNA such as genomic DNA) that includes a site (“target site” or “target sequence”) targeted by a modified CRISPR/Cas effector polypeptide of the present disclosure. The target sequence is the sequence to which the guide sequence of a guide nucleic acid (e.g., guide RNA; e.g., a dual guide RNA or a single-molecule guide RNA) will hybridize. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a viral translation element” includes a plurality of such viral translation elements and reference to “the CRISPR/Cas effector polypeptide” includes reference to one or more CRISPR/Cas effector polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for testing whether a target protein (e.g., a host cell protein) regulates viral RNA translation. The methods comprise: a) introducing into a test host cell comprising a catalytically inactive or a catalytically active CRISPR/Cas effector polypeptide a reporter nucleic acid comprising a nucleotide sequence encoding a bicistronic translation monitor and a regulatory nucleic acid comprising a nucleotide sequence encoding a single guide RNA (sgRNA) that comprises a targeting sequence that specifically binds to a target sequence within a nucleic acid encoding the target protein; and b) detecting expression of the reporter proteins to determine whether a target protein regulates translation via a Cap-dependent element or a Cap-independent element based on the expression of the reporter proteins in the test host cell as compared to the expression in the control host cell. The present disclosure provides kits for performing such methods and comprise: a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and b) a regulatory nucleic acid comprising a nucleotide sequence encoding a single guide RNA (sgRNA) that comprises a targeting sequence that specifically binds to a target sequence within a nucleic acid encoding the target protein.

The compositions and methods disclosed herein can be used to identify new therapeutics targets or repurposed drug targets for blocking viral RNA translation. The compositions and methods can also be used to identify important domains within target proteins that are required for regulating (viral RNA translation) and can inform drug design and development for treating RNA viruses.

The disclosure relates to a method for identifying treatment targets for the treatment of RNA viruses. The method comprises the steps of: (a) generating cells harboring bicistronic reporter expressing cap-dependent or cap-independent viral RNAs of interest; (b) stably introducing catalytically active or catalytically inactive CRISPR/Cas polypeptide in the cells; (c) inhibiting expression of the target gene in the cells generated in step (b) by stably introducing an sgRNA expression construct directed to the target gene of interest; (d) monitoring the host and viral translation in the cells following inhibition of target gene expression; and (f) designating the host and viral translation signals as a target gene knockdown-related condition, if improvement of the condition is observed following step (c).

The disclosure also relates to a method for identifying effect domains of treatment targets relating to RNA viruses. The method comprises the steps of: (a) generating cells harboring a bicistronic reporter expressing cap-dependent or cap-independent viral RNAs of interest; (b) stably introducing catalytically active or catalytically inactive CRISPR/Cas polypeptide in the cells; (c) disrupting sequences in domains of the target gene in the cells generated in step (b) by stably introducing an sgRNA expression construct directed to a domain within a target gene of interest; (d) monitoring the host and viral translation in the cells following disruption of target gene domains; and (f) designating the host and viral translation signals as a target gene domain disruption-related condition, if improvement of the condition is observed following step (c).

The CRISPR system suitable for use in the methods of the present disclosure can be: CRISPR (active Cas9), CRISPRi (CRISPR interference, a catalytically dead Cas9 fused to a transcriptional repressor peptide including KRAB), CRISPRa (CRISPR activation, a catalytically dead Cas9 fused to a transcriptional activator peptide including VPR).

The present disclosure provides a method for testing whether a target protein regulates viral RNA translation, the method comprising:

a) introducing into a host cell:

i) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element;

ii) a regulatory nucleic acid comprising a nucleotide sequence encoding a single guide RNA (sgRNA) that comprises a targeting sequence that specifically binds to a target sequence within a nucleic acid encoding the target protein,

wherein the host cell comprises a catalytically inactive CRISPR/Cas effector polypeptide, thereby generating a test host cell; and

b) detecting expression of the first reporter protein and the second reporter protein in the test host cell and in a control host cell, wherein the control host cell comprises the reporter nucleic acid but not the regulatory nucleic acid,

wherein a target protein is considered to regulate translation via the Cap-dependent element if the expression of the first reporter protein in the test host cell is different compared to the expression of the first reporter protein in the control host cell, and

wherein a target protein is considered to regulate translation via the Cap-independent element if the expression of the second reporter protein in the test host cell is different compared to the expression of the second reporter protein in the control host cell.

Certain embodiments of the present disclosure also provide a method for testing whether a target protein regulates viral RNA translation, the method comprising:

a) introducing into a host cell:

i) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence a second reporter protein translated under the control of a Cap-independent translation element,

ii) a regulatory nucleic acid comprising a nucleotide sequence encoding a single guide RNA (sgRNA) that comprises a targeting sequence that specifically binds to a target sequence within a nucleic acid encoding the target protein,

wherein the host cell comprises a catalytically active CRISPR/Cas effector polypeptide, thereby generating a test host cell; and

b) detecting expression of the first reporter protein and the second reporter protein in the test host cell and in a control host cell, wherein the control host cell comprises the reporter nucleic acid but not the regulatory nucleic acid,

wherein a target protein is considered to regulate translation via the Cap-dependent element if the expression of the first reporter protein in the test host cell is different compared to the expression of the first reporter protein in the control host cell, and

wherein a target protein is considered to regulate translation via the Cap-independent element if the expression of the second reporter protein in the test host cell is different compared to the expression of the second reporter protein in the control host cell.

The present disclosure provides a method for testing whether a target protein regulates viral RNA translation, the method comprising:

a) introducing into a host cell:

i) a reporter nucleic acid comprising a nucleotide sequence encoding a translation monitor, the translation monitor comprising a reporter nucleotide sequence encoding a reporter protein translated under the control of a viral RNA translation element,

ii) a regulatory nucleic acid comprising a nucleotide sequence encoding a single guide RNA (sgRNA) that comprises a targeting sequence that specifically binds to a target sequence within the nucleic acid encoding the target protein,

wherein the host cell comprises a catalytically inactive CRISPR/Cas effector polypeptide, thereby generating a test host cell; and

b) detecting expression of the reporter protein in the test host cell and in a control host cell, wherein the control host cell comprises the reporter nucleic acid but not the regulatory nucleic acid,

wherein a target protein is considered to regulate translation via the viral RNA translation element if the expression of the reporter protein in the test host cell is different compared to the expression of the reporter protein in the control host cell.

Even further embodiments of the present disclosure provide a method for testing whether a target protein regulates viral RNA translation, the method comprising:

a) introducing into a host cell:

i) a reporter nucleic acid comprising a nucleotide sequence encoding a translation monitor, the translation monitor comprising a reporter nucleotide sequence encoding a reporter protein translated under the control a viral RNA translation element,

ii) a regulatory nucleic acid comprising a nucleotide sequence encoding a single guide RNA (sgRNA) that comprises a targeting sequence that specifically binds to a target sequence within a nucleic acid encoding the target protein,

wherein the host cell comprises a catalytically active CRISPR/Cas effector polypeptide, thereby generating a test host cell; and

b) detecting expression of the reporter protein in the test host cell and in a control host cell, wherein the control host cell comprises the reporter nucleic acid but not the regulatory nucleic acid,

wherein a target protein is considered to regulate translation via the viral RNA translation element if the expression of the reporter protein in the test host cell is different compared to the expression of the reporter protein in the control host cell.

Viruses infect cells and use the cellular translation machinery to produce proteins for its multiplication. Viral genetic material uses many types of translation regulatory elements to force the host cellular machinery into translating the viral proteins. Such translation regulatory elements include Cap-dependent elements as well as Cap-independent elements, such a Internal Ribosome Entry Site (IRES) or 5′ untranslated translational regulatory element. Additional Cap-dependent or Cap-independent translation regulatory elements are known in the art and can be tested according to the compositions and methods disclosed herein. Jaffar et al. (2019), Viral RNA structure-based strategies to manipulate translation, Nat. Rev. Microbiol., 17(2):110-123, describe various mechanism of viral RNA translation. The contents of Jaffar et al. are incorporated herein by reference in their entirety.

In certain embodiments, a bicistronic translation monitor used in the methods disclosed herein comprises a first reporter nucleotide sequence encoding a first reporter protein translated under the control of a Cap-dependent translation element and a second reporter nucleotide sequence encoding a second reporter protein translated under the control of a Cap-independent translation element. One or both of the Cap-dependent translation element or the Cap-independent translation element is from a virus. In some cases, only one of the Cap-dependent translation element or the Cap-independent translation element is from a virus and the other translation element is from the host cell. Thus, translation of the reporter protein under the control of the translation element from the host cell can act as an internal control for determining that a target protein only affects the translation of the reporter protein under the control of the viral translation element without affecting the translation of the reporter protein under the control of the host cell translation element.

In certain instances, a monocistronic translation monitor is used, which comprises a reporter nucleotide sequence encoding a reporter protein translated under the control of a Cap-dependent translation element or a Cap-independent translation element from a virus. When monocistronic translation monitor is used, only one reporter protein is produced in the cell and the level of the reporter protein can be used to determine whether a target protein regulates the translational element present in the monocistronic translation monitor. For example, if upon inhibition of the expression of a target protein, the expression of a reporter protein is reduced, the target protein facilitates translation via the translation element. On the other hand, if upon increased expression of a target protein, the expression of a reporter protein is reduced, the target protein inhibits translation via the translation element. Conversely, if upon inhibition of the expression of a target protein, the expression of a reporter protein is increased, the target protein inhibits translation via the translation element. On the other hand, if upon increased expression of a target protein, the expression of a reporter protein is increased, the target protein facilitates translation via the translation element.

The viral translation elements tested according to the methods of the present disclosure can be from a DNA virus or RNA virus. In exemplary embodiments, the viral translation element is from an RNA virus.

The virus can be Adenoviridae (e.g., adenovirus), Arenaviridae (e.g., Machupo virus), Bunyaviridae (e.g., Hantavirus or Rift Valley fever virus), Coronaviridae, Orthomyxoviridae (e.g., influenza viruses), Filoviridae (e.g., Ebola virus and Marburg virus), Flaviviridae (e.g., Japanese encephalitis virus; Hepatitis C Virus; and Yellow fever virus), Hepadnaviridae (e.g., hepatitis B virus), Herpesviridae (e.g., herpes simplex viruses), Papovaviridae (e.g., papilloma viruses), Paramyxoviridae (e.g., respiratory syncytial virus, measles virus, mumps virus, or parainfluenza virus), Parvoviridae, Picornaviridae (e.g., polioviruses), Poxviridae (e.g., variola viruses), Reoviridae (e.g., rotaviruses), Retroviridae (e.g., human T cell lymphotropic viruses (HTLV) and human immunodeficiency viruses (HIV)), Rhabdoviridae (e.g., rabies virus), and Togaviridae (e.g., encephalitis viruses, yellow fever virus, and rubella virus)). Additional examples of RNA or DNA viruses that could be tested according to the methods disclosed herein are well known in the art and such embodiments are within the purview of the present disclosure.

The host cell comprises a catalytically inactive CRISPR/Cas effector polypeptide. Therefore, such host cell, when transfected with a nucleic acid comprising an appropriate nucleotide sequence encoding an sgRNA comprising a targeting sequence specific for (i.e. complementary to) a nucleotide sequence encoding a target protein, the transcription of the mRNA encoding the target protein is inhibited.

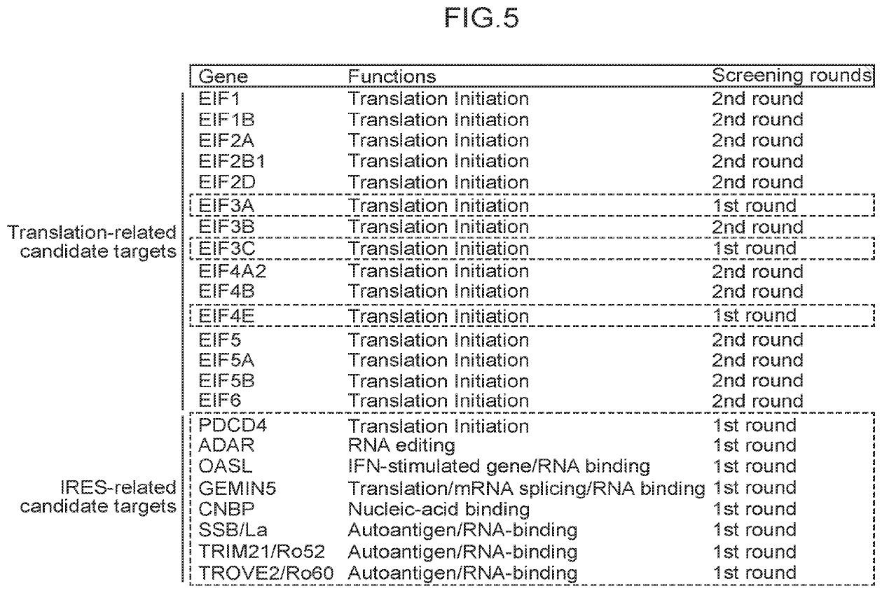

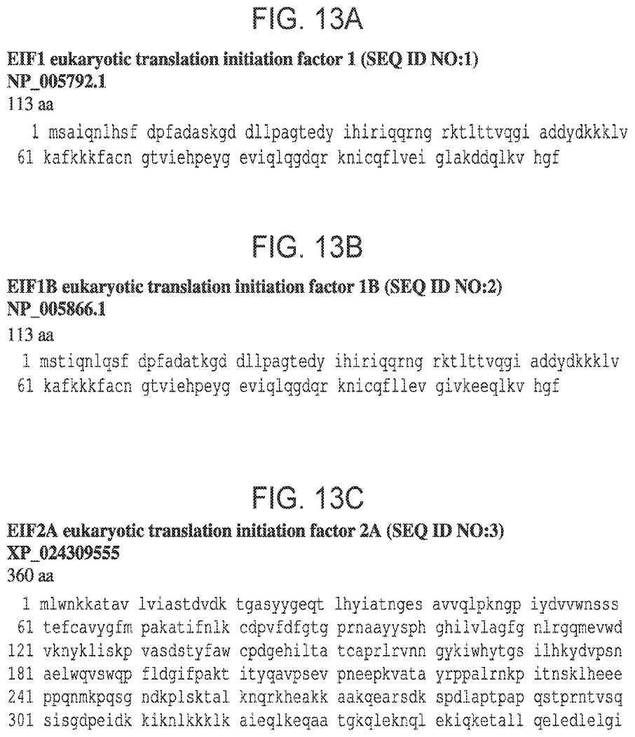

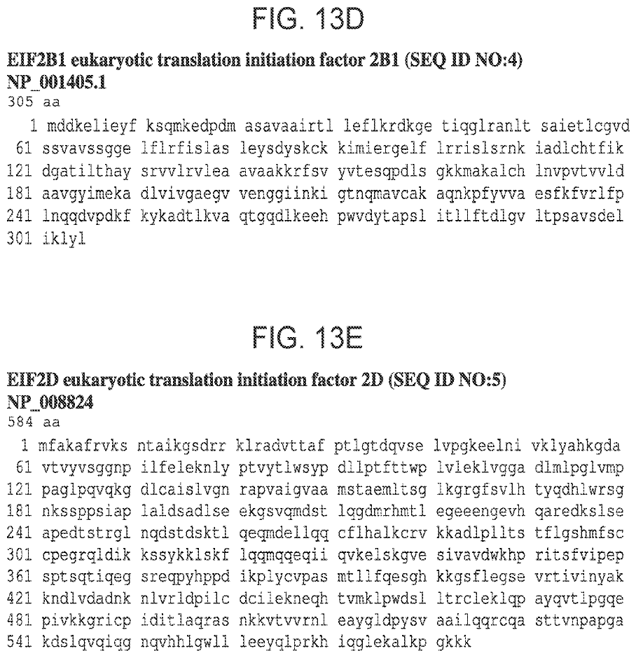

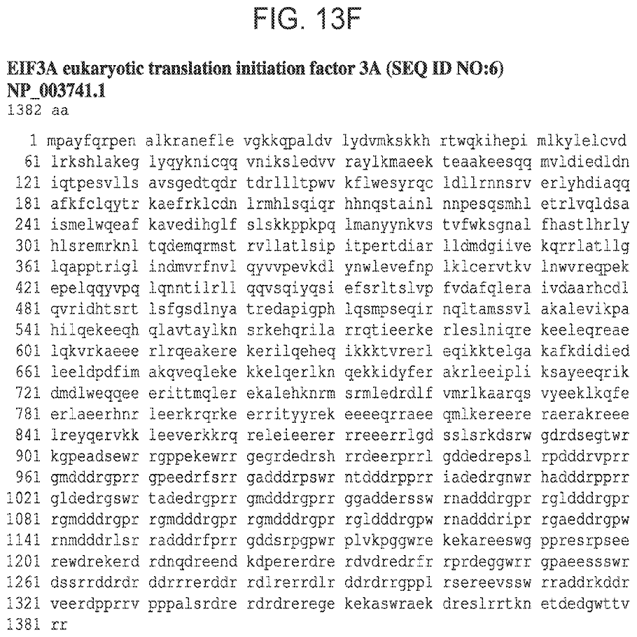

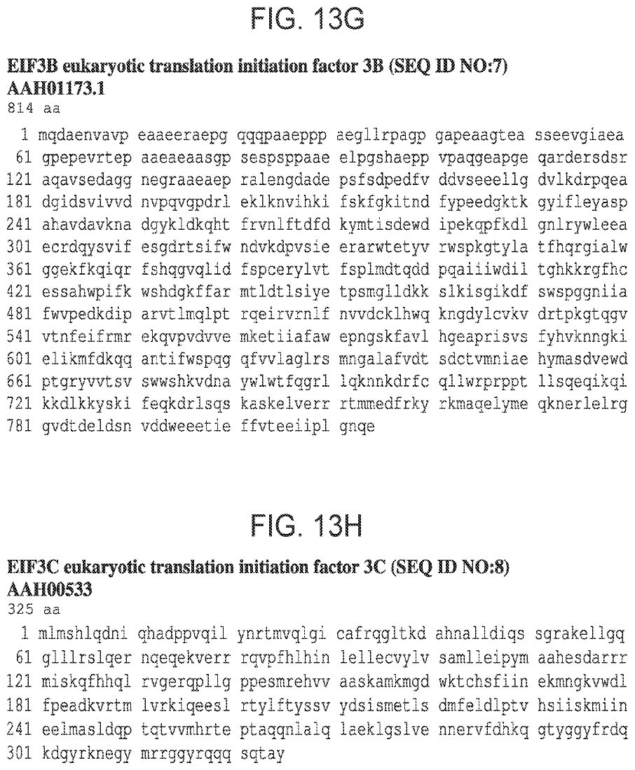

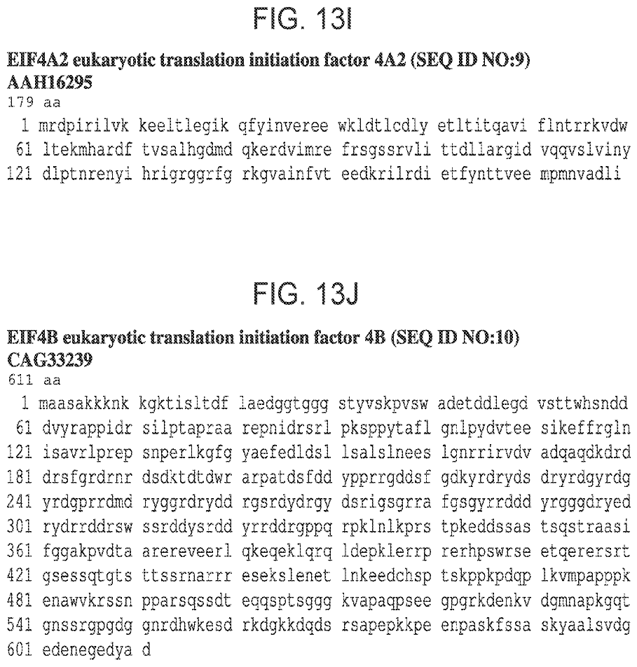

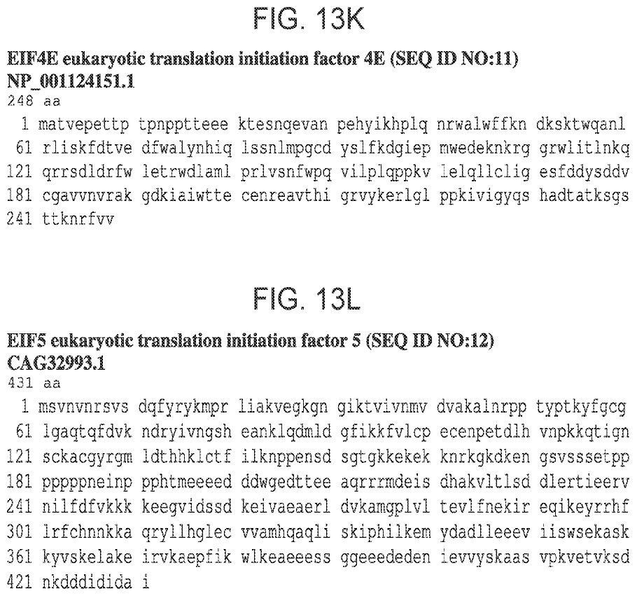

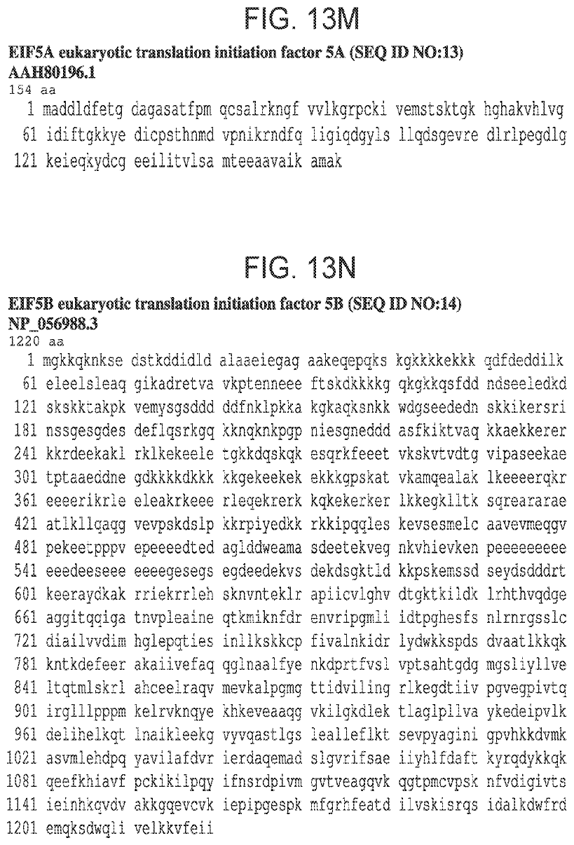

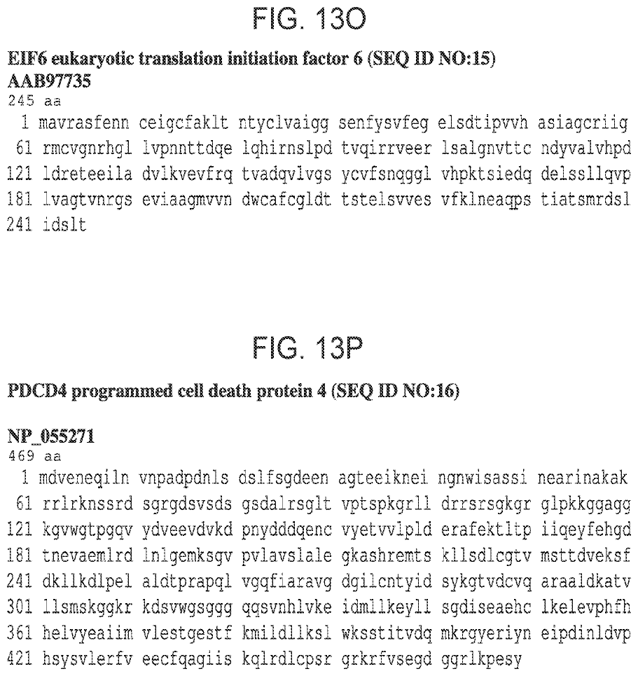

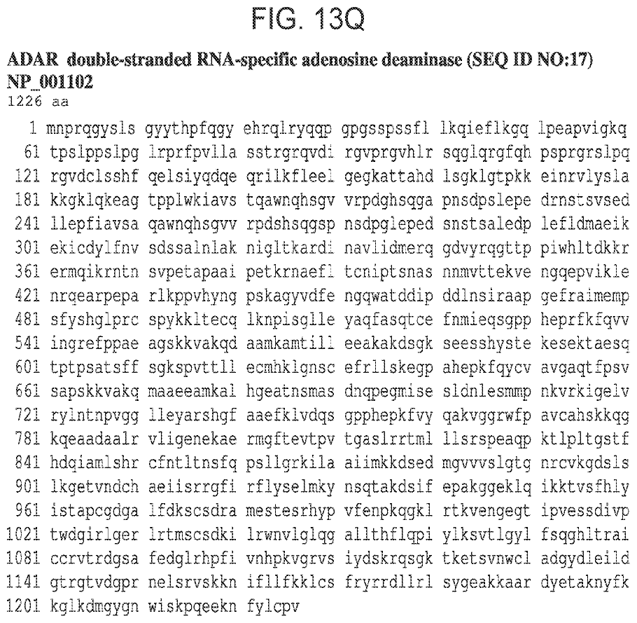

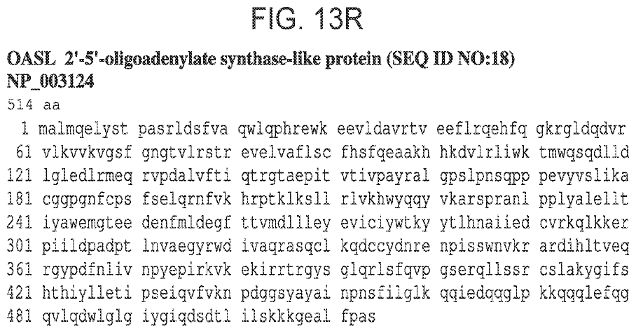

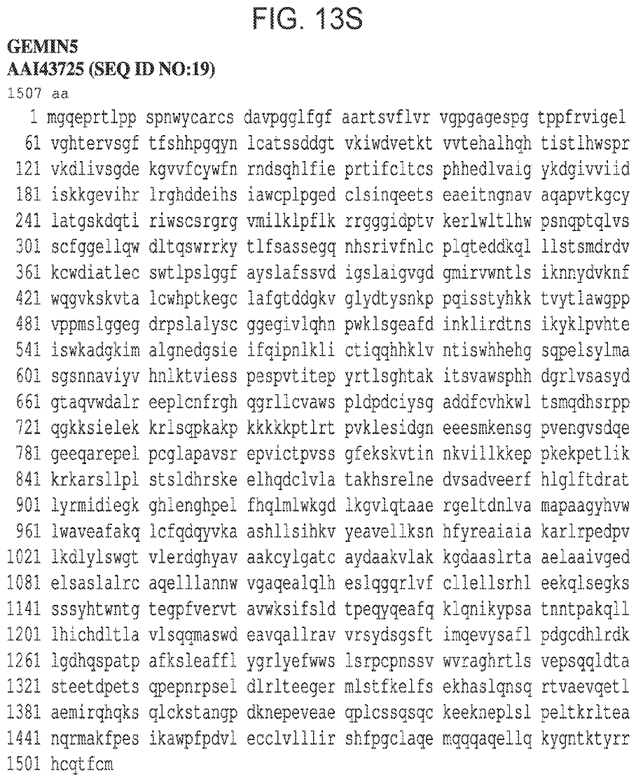

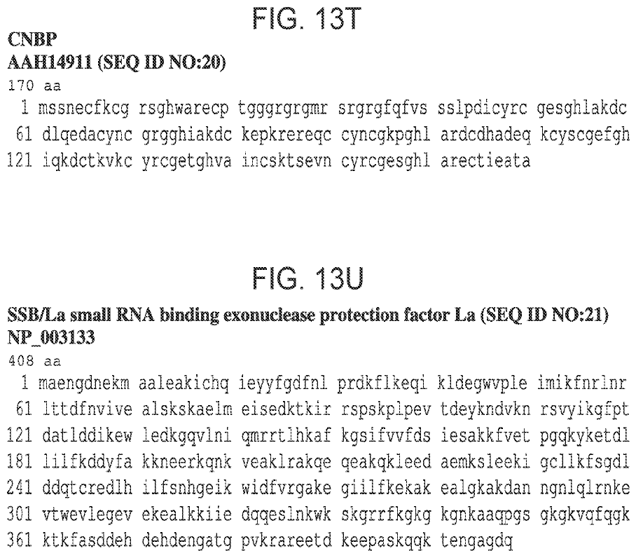

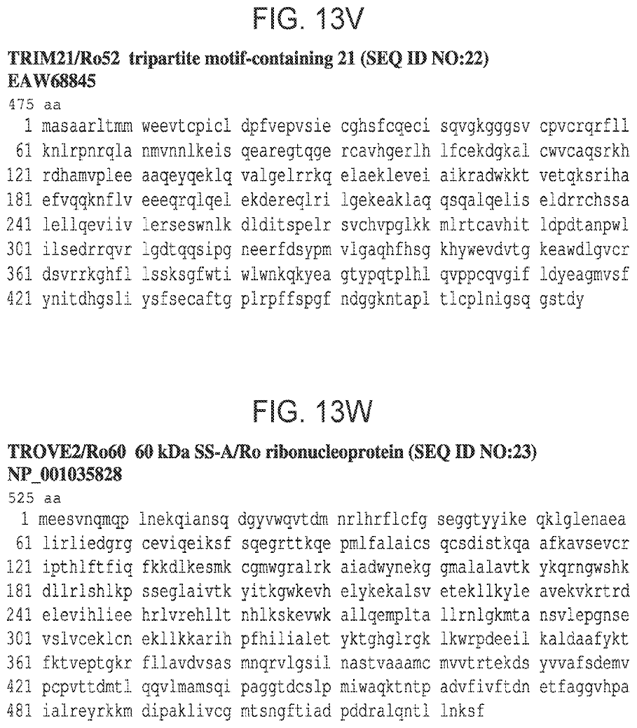

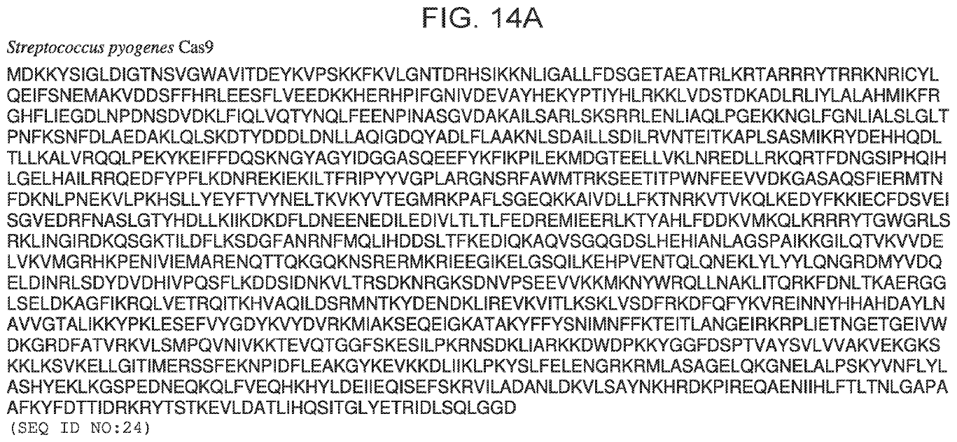

The target protein can be any protein; e.g., a protein suspected of regulating translation. Non-limiting examples of such proteins are provided in FIG. 5. Sequences of exemplary target proteins are provided in FIGS. 13A-13W. The target protein is a host cell-encoded protein; e.g., a protein encoded by a mammalian host cell that can be infected with an RNA virus. The target protein can be a mammalian protein, e.g., a human protein.

Any host cell can be used in the methods of the present disclosure and the selection of the host cell depends on the virus being studied and its natural host. For example, for studying a virus infecting human liver cells the host cell can be primary or secondary human liver cells, such as Hep G2. Additional cells that can be used in the methods disclosed herein include 3T3-L1, C2C12, CHO, COS-7, HEK293, HEK293T, HeLa, Hepa1C1c7, Hep G2, Jurkat, MRC-5, NIH-3T3, Raji, and Vero. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. Even further cells that can be used in the methods disclosed herein are known in the art and such embodiments are within the purview of the present disclosure.

As noted above, the host cell comprises a catalytically inactive or catalytically active CRISPR/Cas effector polypeptide. Such host cell can be produced by transiently or permanently transfecting the host cell with a nucleic acid comprising a nucleotide sequence encoding the catalytically inactive CRISPR/Cas effector polypeptide. CRISPR/Cas effector polypeptides

Any CRISPR/Cas effector polypeptide is suitable for use in the methods disclosed herein. In some cases, the CRISPR/Cas effector polypeptide used in the methods disclosed herein is a type II CRISPR/Cas effector polypeptide, a type V CRISPR/Cas effector polypeptide, or a type VI CRISPR/Cas effector polypeptide. In some cases, the CRISPR/Cas effector polypeptide used in the methods disclosed herein is a type II CRISPR/Cas effector polypeptide. In some cases, the CRISPR/Cas effector polypeptide used in the methods disclosed herein is a Cas9 polypeptide. In some cases, the CRISPR/Cas effector polypeptide used in the methods disclosed herein is a type V CRISPR/Cas effector polypeptide. In some cases, the CRISPR/Cas effector polypeptide used in the methods disclosed herein is a Cas12a, a Cas12b, a Cas12c, a Cas12d, or a Cas12e polypeptide. In some cases, the CRISPR/Cas effector polypeptide used in the methods disclosed herein is a type VI CRISPR/Cas effector polypeptide. In some cases, the CRISPR/Cas effector polypeptide used in the methods disclosed herein is a Cas13a, a Cas13b, a Cas13c, or a Cas13d polypeptide. In some cases, the CRISPR/Cas effector polypeptide used in the methods disclosed herein is a Cas14a, a Cas14b, or a Cas14c polypeptide Amino acid sequences of a variety of CRISPR/Cas effector polypeptides are known.

Examples of various Cas9 proteins (and Cas9 domain structure) and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci U S A. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protocols 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci U S A. 2013 Sep 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; Shmakov et al., Nat Rev Microbiol. 2017 March; 15(3):169-182; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

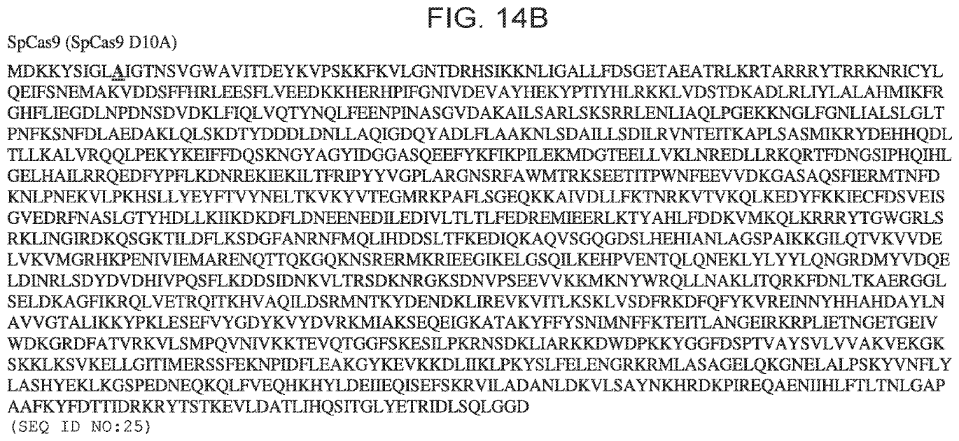

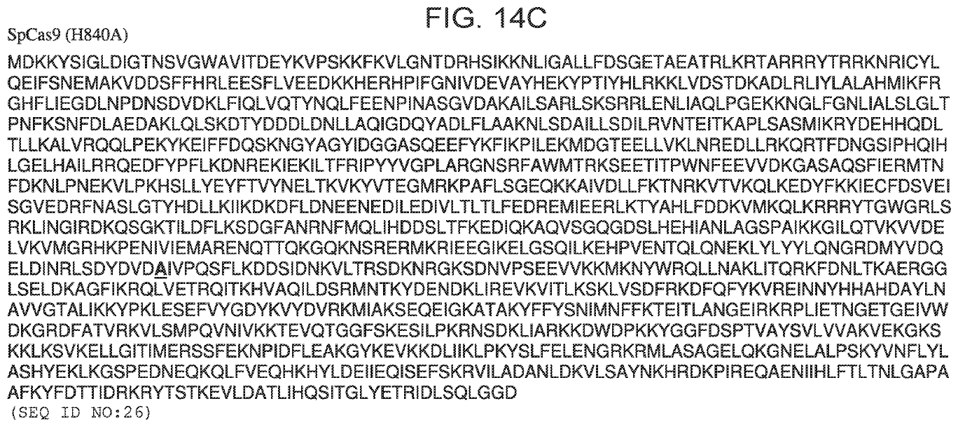

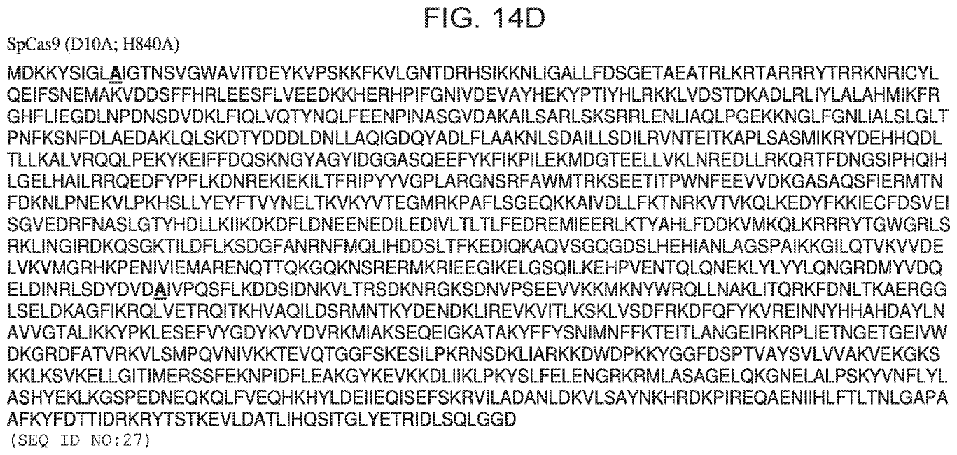

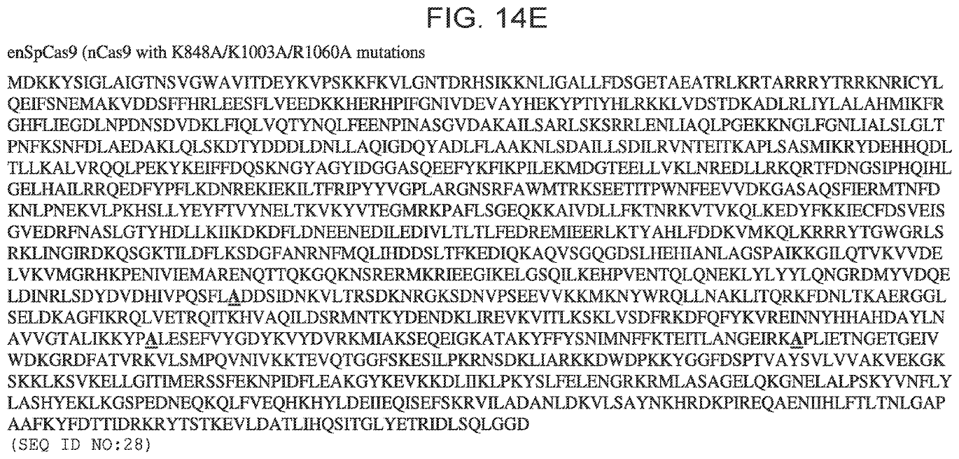

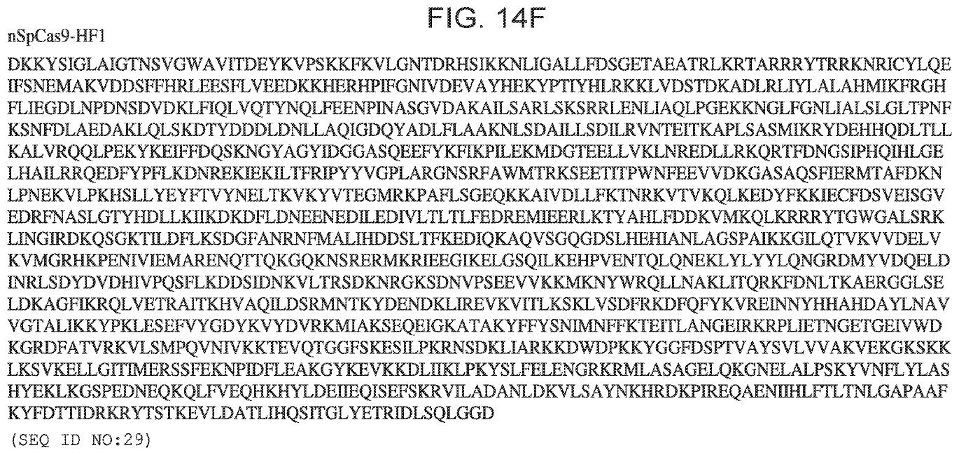

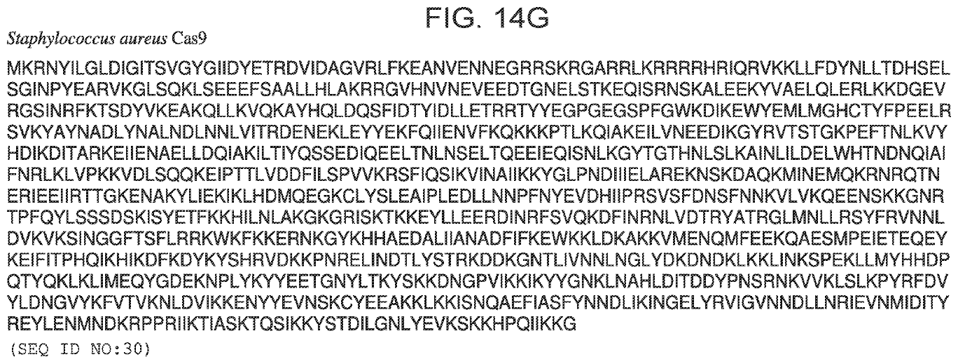

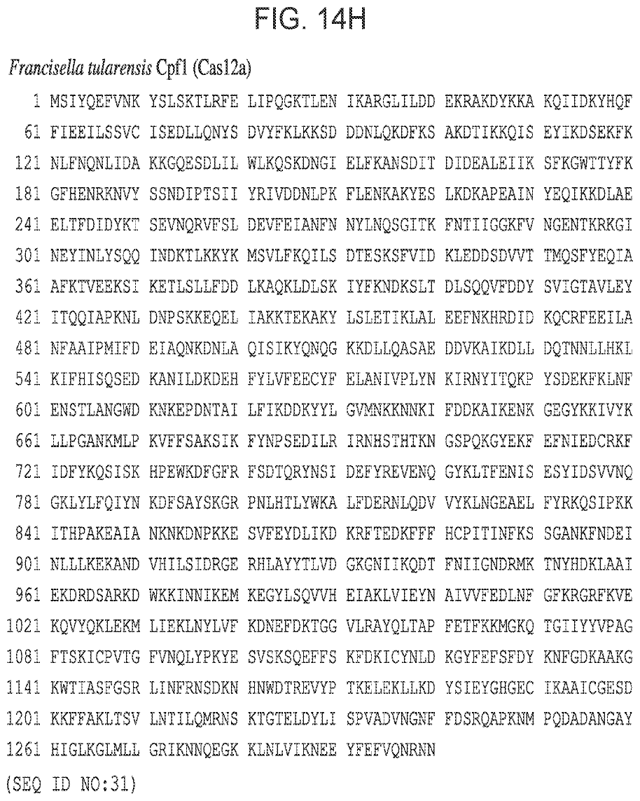

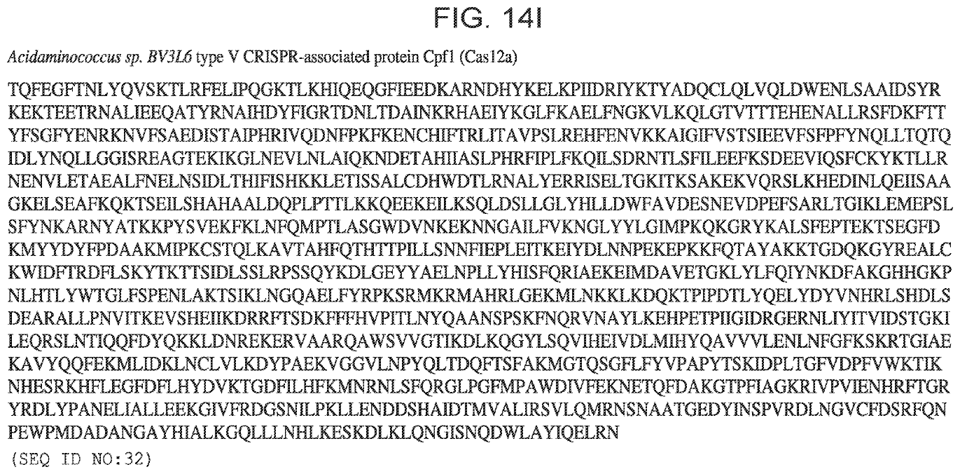

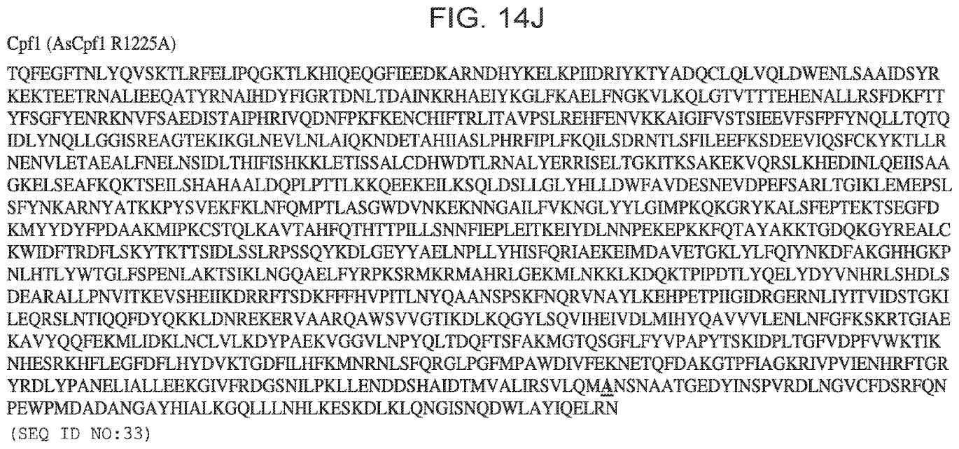

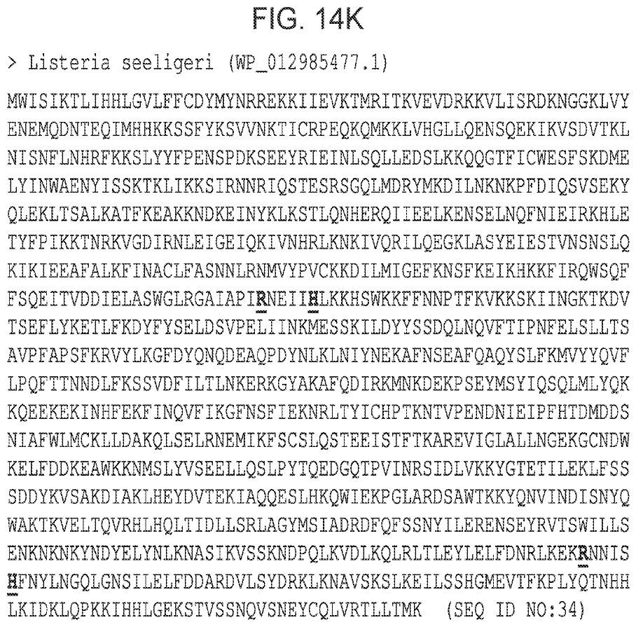

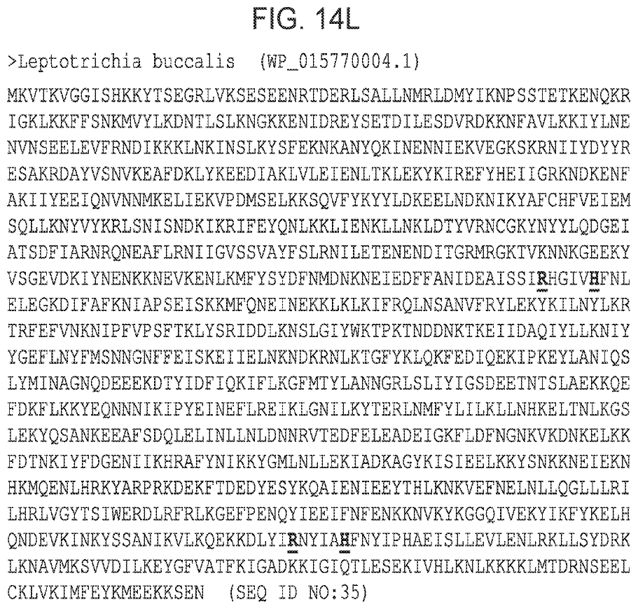

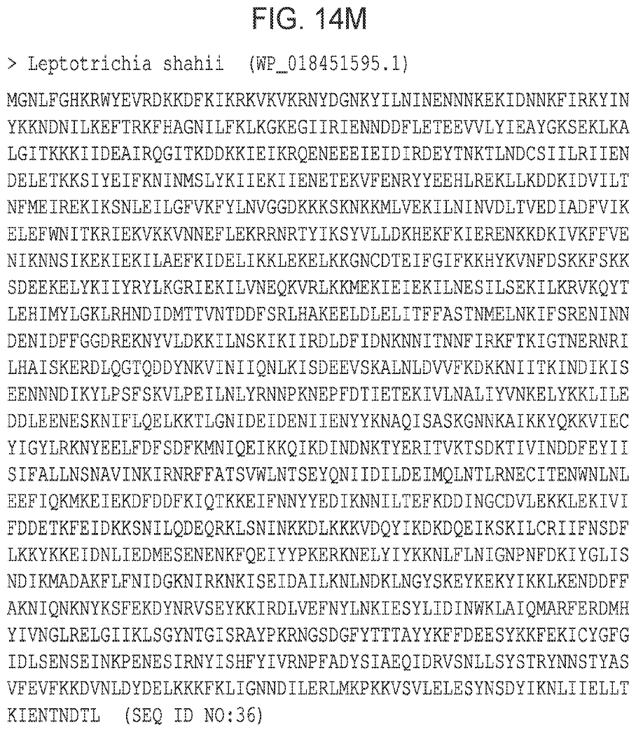

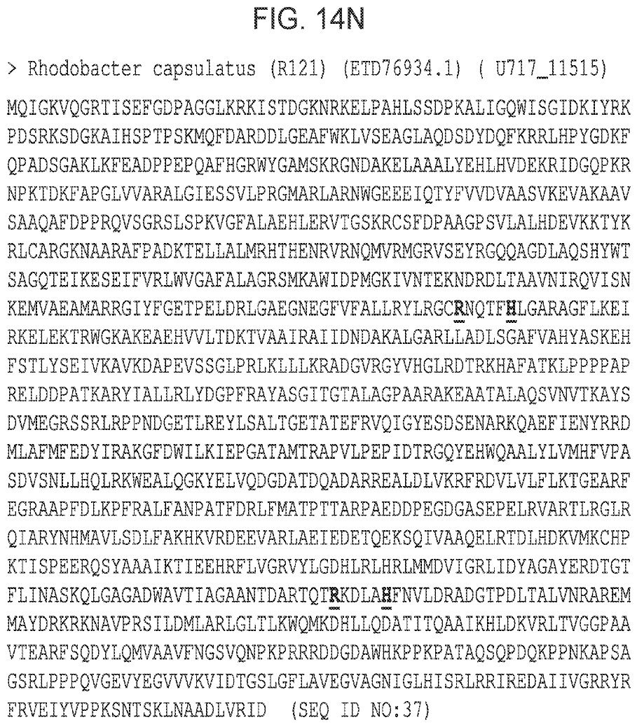

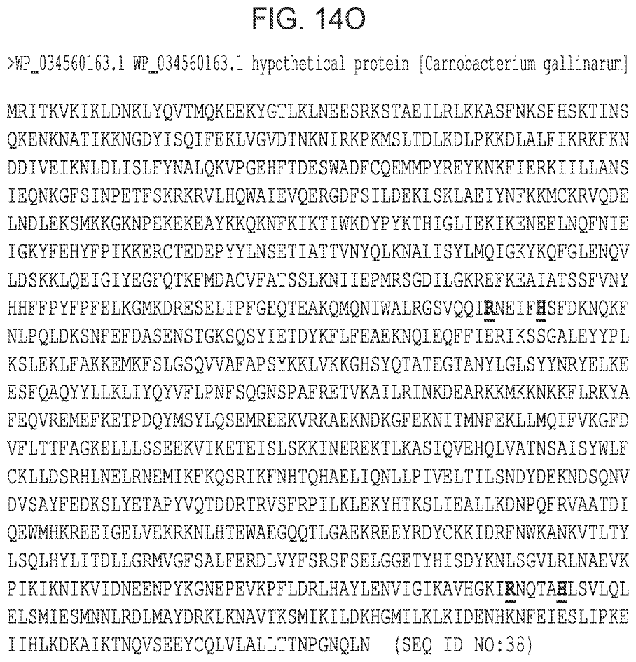

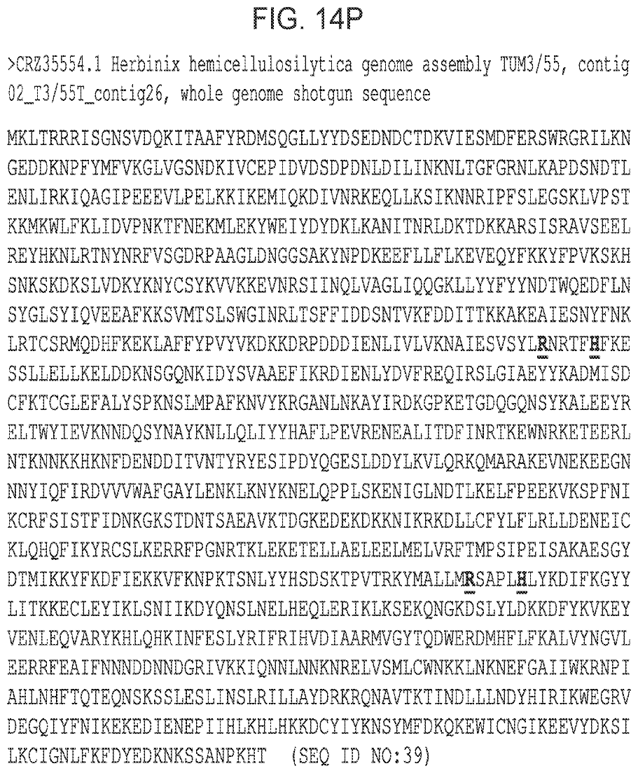

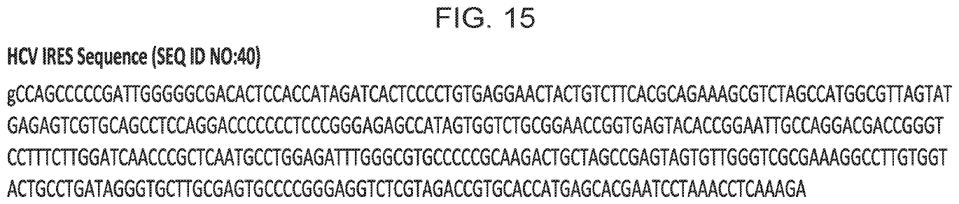

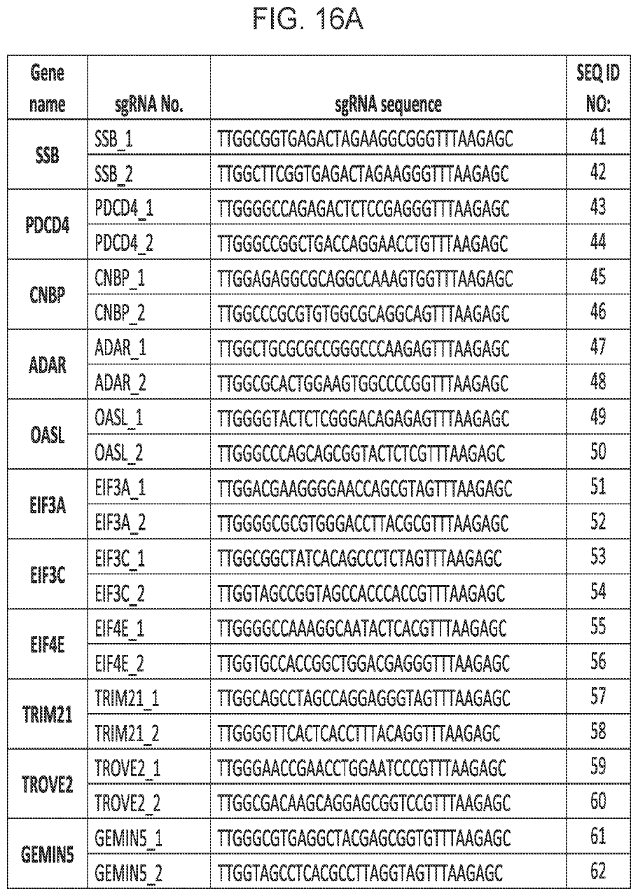

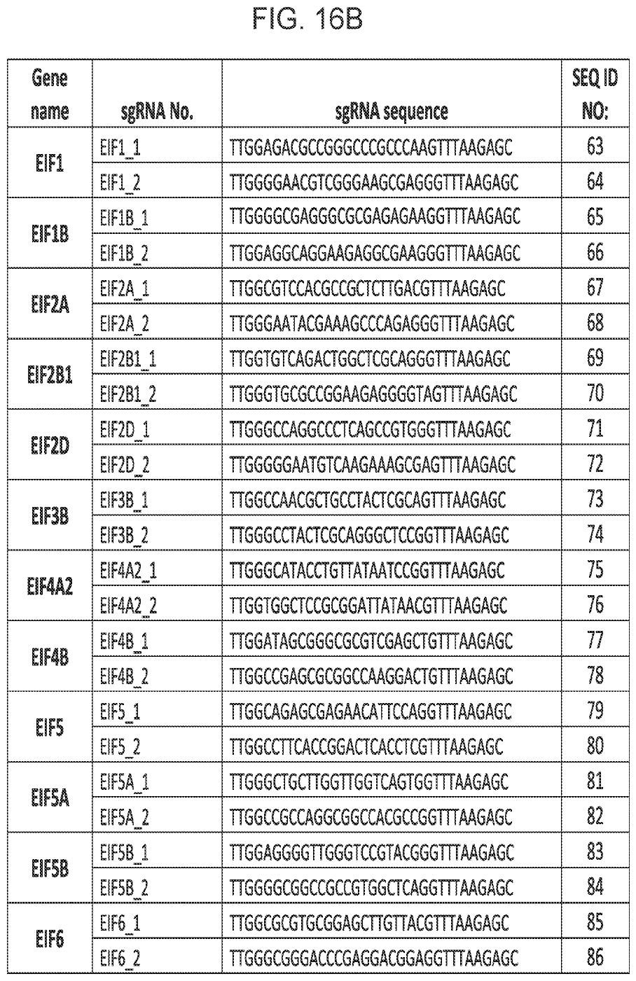

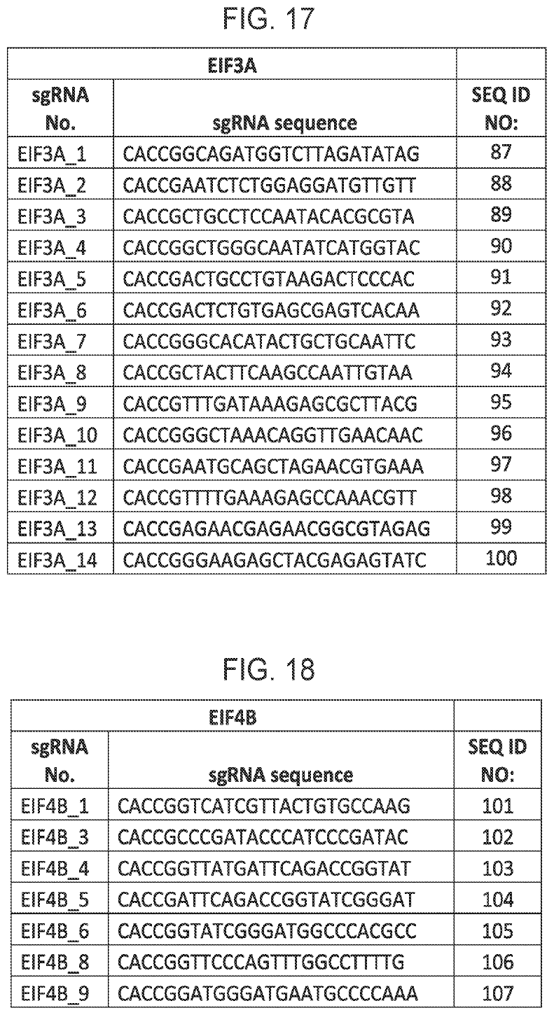

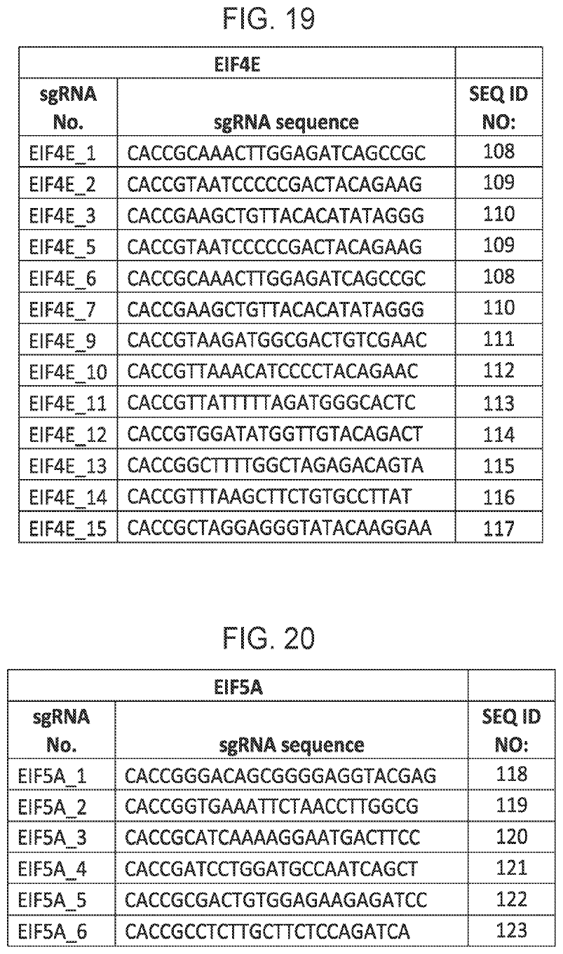

In some cases, a CRISPR/Cas effector polypeptide used in the methods disclosed herein comprises an amino acid sequence having at least 85%, at least 90%, at least 95%, at least 98%, at least 99%, or 100%, amino acid sequence identity to the amino acid sequence depicted in any one of FIG. 14A-14P.

In some cases, a CRISPR/Cas effector polypeptide used in the methods disclosed herein is enzymatically active. In some cases, a CRISPR/Cas effector polypeptide used in the methods disclosed herein exhibits reduced enzymatic activity compared to a wild-type CRISPR/Cas effector polypeptide. In some cases, a CRISPR/Cas effector polypeptide used in the methods disclosed herein is a nickase. For example, in some cases, a CRISPR/Cas effector polypeptide used in the methods disclosed herein comprises a substitution of D10 (e.g., D10A) or H840 (e.g., H840A) of the amino acid sequence depicted in FIG. 14A, or a corresponding amino acid of another CRISPR/Cas effector polypeptide. In some cases, a CRISPR/Cas effector polypeptide used in the methods disclosed herein is enzymatically inactive (a “dead” CRISPR/Cas effector polypeptide) but retains the ability to bind a target nucleic acid when complexed with a guide nucleic acid. For example, in some cases, a CRISPR/Cas effector polypeptide used in the methods disclosed herein comprises a substitution of both D10 and H840 (e.g., D10A; and H840A) of the amino acid sequence depicted in FIG. 14A, or corresponding amino acids of another CRISPR/Cas effector polypeptide.

A catalytically inactive CRISPR/Cas effector polypeptide inhibits transcription of the target nucleic acid based on the binding of the sgRNA to the target nucleic acid. Under such conditions, the target protein is not present or is present in a lower amount in the host cell. Consequently, if the target protein facilitates viral translation, reduced amount of the target protein would inhibit such viral translation. Therefore, in such cell the reduced expression of a reporter protein under the control of the viral translation element would indicate that the target protein facilitates viral translation, whereas, unchanged expression of a reporter protein under the control of the viral translation element would indicate that the target protein is not involved in the regulation of viral translation. Alternatively, the increased expression of a reporter protein under the control of the viral translation element would indicate that the target protein inhibits viral translation.

In certain embodiments, a catalytically inactive CRISPR/Cas effector polypeptide is fused to a transcription regulator, such as a transcription activator or a transcription inhibitory.

When the CRISPR/Cas effector polypeptide is fused to a transcription inhibitor, it inhibits the expression of the target protein. Under such conditions, the target protein is not present or is present in a lower amount in the host. Consequently, if the target protein facilitates viral translation, reduced amount of the target protein would inhibit such viral translation. Therefore, in such cell the reduced expression of a reporter protein under the control of the viral translation element would indicate that the target protein facilitates viral translation, whereas, unchanged expression of a reporter protein under the control of the viral translation element would indicate that the target protein is not involved in the regulation of viral translation. Alternatively, the increased expression of a reporter protein under the control of the viral translation element would indicate that the target protein inhibits viral translation.

Non-limiting examples of transcription inhibitors that can be fused to the catalytically inactive CRISPR/Cas effector polypeptides include Krüppel associated box (KRAB), MAX Interactor 1, Dimerization Protein (Mxi1), Transcriptional repressor TUP1 (TUP1), Regulatory protein MIG1 (MIG1), Crt 1 transcription factor (CRT1), extra cotyledon 1 (XTC1), Unscheduled Meiotic gene Expression (UME6). Additional embodiments of transcription inhibitors suitable for use in the inactive CRISPR/Cas effector polypeptides for use in the methods disclosed herein are known in the art and such embodiments are within the purview of the present disclosure.

When the CRISPR/Cas effector polypeptide is fused to a transcription activator, it induces the expression of the target protein. Under such conditions, the target protein is present in a higher amount in the host. Consequently, if the target protein facilitates viral translation, increased amount of the target protein would increase such viral translation. Therefore, in such cell the increased expression of a reporter protein under the control of the viral translation element would indicate that the target protein facilitates viral translation, whereas, unchanged expression of a reporter protein under the control of the viral translation element would indicate that the target protein is not involved in the regulation of viral translation. Alternatively, the reduced expression of a reporter protein under the control of the viral translation element would indicate that the target protein inhibits viral translation.

Non-limiting examples of transcription activators that can be fused to the catalytically inactive CRISPR/Cas effector polypeptides include VP64, Transactivating tegument protein VP16 (VP16), activation domain VPR (composed of the activation domains of VP64, P65, and Rta), NFØB transcription factor p65 (p65AD), and viral Rta protein (Rta). Additional embodiments of transcription activators suitable for use in the inactive CRISPR/Cas effector polypeptides for use in the methods disclosed herein are known in the art and such embodiments are within the purview of the present disclosure.

In certain embodiments, a catalytically active CRISPR/Cas effector polypeptide is used in the methods disclosed herein. A catalytically active CRISPR/Cas effector polypeptide would inhibit the expression of the target nucleic acid based on the binding of the sgRNA to the target nucleic acid. Under such conditions, the target protein is not present or is present in a lower amount in the host cell. Consequently, if the target protein facilitates viral translation, reduced amount of the target protein would inhibit such viral translation. Therefore, in such cell the reduced expression of a reporter protein under the control of the viral translation element would indicate that the target protein facilitates viral translation, whereas, unchanged expression of a reporter protein under the control of the viral translation element would indicate that the target protein is not involved in the regulation of viral translation. Alternatively, the increased expression of a reporter protein under the control of the viral translation element would indicate that the target protein inhibits viral translation.

In certain embodiments, a catalytically active CRISPR/Cas effector polypeptide is used in the methods disclosed herein in combination with an sgRNA that specifically binds to a within the nucleic acid encoding target protein at a nucleotide sequence that encodes a domain of the target protein. In these conditions, a target protein without the targeted domain is synthesized in the cells. Consequently, if the target domain of the target protein facilitates viral translation, reduced amount of the target protein containing the target domain would inhibit such viral translation. Therefore, in such cell the reduced expression of a reporter protein under the control of the viral translation element would indicate that the target domain of the target protein facilitates viral translation, whereas, unchanged expression of a reporter protein under the control of the viral translation element would indicate that the target domain of the target protein is not involved in the regulation of viral translation. Alternatively, the increased expression of a reporter protein under the control of the viral translation element would indicate that the target domain of the target protein inhibits viral translation.

### Guide Nucleic Acid

A guide nucleic acid suitable for inclusion in a system of the present disclosure can include: i) a first segment (referred to herein as a “targeting segment”); and ii) a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. The “targeting segment” is also referred to herein as a “variable region” of a guide RNA. The “protein-binding segment” is also referred to herein as a “constant region” of a guide RNA. The first segment (targeting segment) of a guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a CRISPR/Cas effector polypeptide. The protein-binding segment of a guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the guide RNA (the guide sequence of the guide RNA) and the target nucleic acid.

A guide RNA and a CRISPR/Cas effector polypeptide form a complex (e.g., bind via non-covalent interactions). The guide RNA provides target specificity to the complex by including a targeting segment, which includes a guide sequence (a nucleotide sequence that is complementary to a sequence of a target nucleic acid). The CRISPR/Cas effector polypeptide of the complex provides the site-specific activity (e.g., cleavage activity or an activity provided by the CRISPR/Cas effector polypeptide when the CRISPR/Cas effector polypeptide is a CRISPR/Cas effector polypeptide fusion polypeptide, i.e., has a fusion partner). In other words, the CRISPR/Cas effector polypeptide is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the guide RNA.

The “guide sequence” also referred to as the “targeting sequence” of a guide RNA can be modified so that the guide RNA can target a CRISPR/Cas effector polypeptide to any desired sequence of any desired target nucleic acid, with the exception that the protospacer adjacent motif (PAM) sequence can be considered. Thus, for example, a guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some cases, a guide RNA includes two separate nucleic acid molecules: an “activator” and a “targeter” and is referred to herein as a “dual guide RNA”, a “double-molecule guide RNA”, or a “two-molecule guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

Examples of various CRISPR/Cas effector polypeptides and guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci U S A. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 Octpber; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 Dec; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci U S A. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; Shmakov et al., Nat Rev Microbiol. 2017 Mar; 15(3):169-182; and U.S. patents and patent applications: U.S. Pat. Nos.8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

In some cases, a guide nucleic acid comprises ribonucleotides only, deoxyribonucleotides only, or a mixture of ribonucleotides and deoxyribonucleotides. In some cases, a guide nucleic acid comprises ribonucleotides only, and is referred to herein as a “guide RNA.” In some cases, a guide nucleic acid comprises deoxyribonucleotides only, and is referred to herein as a “guide DNA.” In some cases, a guide nucleic acid comprises both ribonucleotides and deoxyribonucleotides. A guide nucleic acid can comprise combinations of ribonucleotide bases, deoxyribonucleotide bases, nucleotide analogs, modified nucleotides, and the like; and may further include naturally-occurring backbone residues and/or linkages and/or non-naturally-occurring backbone residues and/or linkages.

In some embodiments, the regulatory nucleic acid comprising a nucleotide sequence encoding the sgRNA also comprises one or more of: i) a reporter nucleic acid comprising a nucleotide sequence encoding a third reporter protein; ii) a selectable marker gene; and iii) a promoter driving the expression of the sgRNA.

A reporter nucleic acid encodes a reporter protein that can be used as a proxy for the expression of the sgRNA. Reporter proteins discussed elsewhere in this disclosure can also be used in the reporter nucleic acid.

The selectable marker gene can be used to exert positive pressure on the cells so that the regulatory nucleic acid is maintained in the cell. As an example, a selectable marker gene encodes a protein that confers resistance in the cells to an antibiotic, such as zeocin, hygromycin, blasticidin, puromycin and geneticin. Examples of additional antibiotics and proteins that confer resistance to such antibiotics are well known in the art and are within the purview of the present disclosure.

Promoter driving the expression of sgRNA ensures that the sgRNA is expressed at a desirable level. Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator.

In some cases, a nucleotide sequence encoding a guide RNA is operably linked to an inducible promoter. In some embodiments, a nucleotide sequence encoding a guide RNA is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

In some cases, a nucleotide sequence encoding a guide RNA is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA in a eukaryotic cell it may sometimes be necessary to modify the sequence encoding the guide RNA to eliminate runs of Ts. In some cases, a nucleotide sequence encoding a fusion polypeptide of the present disclosure is operably linked to a promoter operable in a eukaryotic cell (e.g., a CMV promoter, an EF1α promoter, an estrogen receptor-regulated promoter, and the like).

### Introducing Components Into a Target Cell

A reporter nucleic acid and/or regulatory nucleic acid can be introduced into a host cell by any of a variety of well-known methods.

Methods of introducing a nucleic acid into a host cell are known in the art, and any convenient method can be used to introduce a subject nucleic acid (e.g., an expression construct/vector) into a target cell (e.g., eukaryotic cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like.

In some cases, a nucleic acid is delivered to a cell (e.g., a target host cell) in a particle, or associated with a particle. In some cases, a system of the present disclosure is delivered to a cell in a particle, or associated with a particle. The terms “particle” and “nanoparticle” can be used interchangeably, as appropriate. A recombinant expression vector comprising a nucleotide sequence can be delivered via a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5). For example, a particle can be formed using a multistep process in which a modified CRISPR/Cas effector polypeptide of the present disclosure and a guide RNA are mixed together, e.g., at a 1:1 molar ratio, e.g., at room temperature, e.g., for 30 minutes, e.g., in sterile, nuclease free 1 x phosphate-buffered saline (PBS); and separately, DOTAP, DMPC, PEG, and cholesterol as applicable for the formulation are dissolved in alcohol, e.g., 100% ethanol; and, the two solutions are mixed together to form particles containing the complexes).

Lipidoid compounds (e.g., as described in US patent application 20110293703) are also useful in the administration of polynucleotides, and can be used to deliver a reporter nucleic acid and/or regulatory nucleic acid. In one aspect, the aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles.

A poly(beta-amino alcohol) (PBAA) can be used to deliver a nucleic acid of the present disclosure to a target cell. US Patent Publication No. 20130302401 relates to a class of poly(beta-amino alcohols) (PBAAs) that has been prepared using combinatorial polymerization.

Sugar-based particles may be used, for example GalNAc, as described with reference to WO2014118272 (incorporated herein by reference) and Nair, J K et al., 2014, Journal of the American Chemical Society 136 (49), 16958-16961) to deliver a nucleic acid of the present disclosure to a target cell.

In some cases, lipid nanoparticles (LNPs) are used to deliver a nucleic acid of the present disclosure to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(.omega.-methoxy-poly(ethylene glycol)2000) carbamoyl]-1,2-dimyristyloxlpropyl-3-amine (PEG-C-DOMG) may be used. A nucleic acid (e.g., a guide RNA; a nucleic acid of the present disclosure; etc.) may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). In some cases, 0.2% SP-DiOC18 is incorporated.

Spherical Nucleic Acid (SNATM) constructs and other nanoparticles (particularly gold nanoparticles) can be used to deliver a nucleic acid of the present disclosure to a target cell. See, e.g., Cutler et al., J. Am. Chem. Soc. 2011 133:9254-9257, Hao et al., Small 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

In general, a “nanoparticle” refers to any particle having a diameter of less than 1000 nm. In some cases, nanoparticles suitable for use in delivering a nucleic acid of the present disclosure to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a nucleic acid of the present disclosure to a target cell have a diameter of from 25 nm to 200 nm. In some cases, nanoparticles suitable for use in delivering a nucleic acid of the present disclosure to a target cell have a diameter of 100 nm or less. In some cases, nanoparticles suitable for use in delivering a nucleic acid of the present disclosure to a target cell have a diameter of from 35 nm to 60 nm.

Nanoparticles suitable for use in delivering a nucleic acid of the present disclosure to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure.

Semi-solid and soft nanoparticles are also suitable for use in delivering a nucleic acid of the present disclosure to a target cell. A prototype nanoparticle of semi-solid nature is the liposome.

In some cases, an exosome is used to deliver a nucleic acid of the present disclosure to a target cell. Exosomes are endogenous nano-vesicles that transport RNAs and proteins, and which can deliver RNA to the brain and other target organs.

In some cases, a liposome is used to deliver a nucleic acid of the present disclosure to a target cell. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver a nucleic acid of the present disclosure to a target cell. The SNALP formulation may contain the lipids 3-N-[methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N; N-dimethyl)aminopropane (DLinDMA).

Other cationic lipids, such as amino lipid 2,2-dilinoleyl-4-dimethylaminoethyl[1,3]-dioxolane (DLin-KC2-DMA) can be used to deliver a nucleic acid of the present disclosure to a target cell. A preformed vesicle with the following lipid composition may be contemplated amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a system of the present disclosure or component(s) thereof or nucleic acids encoding the same to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a system, or component thereof, of the present disclosure, using a spontaneous vesicle formation procedure. The component molar ratio may be about 50/10/38.5/1.5 (DLin-KC2-DMA or C12-200/disteroylphosphatidyl choline/cholesterol/PEG-DMG).

A reporter nucleic acid and/or regulatory nucleic acid may be delivered encapsulated in PLGA microspheres such as that further described in US published applications 20130252281 and 20130245107 and 20130244279.

Supercharged proteins can be used to deliver a nucleic acid of the present disclosure to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can enable the functional delivery of these macromolecules into mammalian cells both in vitro and in vivo.

Cell Penetrating Peptides (CPPs) can be used to deliver a nucleic acid of the present disclosure to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

### Cells

The present disclosure provides a cell (a “modified cell”) comprising a reporter nucleic acid and/or regulatory nucleic acid of the present disclosure.

A cell that serves as a recipient for a reporter nucleic acid and/or regulatory nucleic acid can be any of a variety of eukaryotic cells (e.g., mammalian cells), including, e.g., in vitro cells; in vivo cells; ex vivo cells; primary cells; cancer cells; animal cells; etc. A cell that serves as a recipient for a reporter nucleic acid and/or regulatory nucleic acid of the present disclosure is referred to as a “host cell” or a “target cell.”

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some cases, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other instances, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other instances, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

Different reporter proteins expressed in a cell can be monitored according to the methods known in the art. Exemplary embodiments of the reporter proteins include is green fluorescent protein, red fluorescent protein, luciferase, β-galactosidase, yellow fluorescent protein, blue fluorescent protein, orange fluorescent protein, or a protein that confers antibiotic resistance, and wherein the first reporter protein is different from the second reporter protein. Additional examples of reporter proteins as well as methods of detecting them in a cell are well-known in the art and such embodiments are within the purview of the present disclosure.

The methods disclosed herein can be used to screen a plurality of target proteins for testing whether one or more of the plurality of target proteins regulate viral RNA translation. Any of the methods disclosed herein could be used for such screening. To screen a plurality of proteins, each of the plurality of proteins is targeted using an sgRNA specific for the protein. As such, a library of regulatory nucleic acids is used, each regulatory nucleic acid comprising a nucleotide sequence encoding a specific sgRNA.

The present disclosure provides kits for carrying out the methods of the present disclosure.

In certain embodiments, the kits disclosed herein comprise:
a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and
b) a regulatory nucleic acid comprising a nucleotide sequence encoding a single guide RNA (sgRNA) that comprises a targeting sequence that specifically binds to a target sequence within a nucleic acid encoding the target protein.

In further embodiments, the kits disclosed herein comprise:

a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and
b) a plurality of regulatory nucleic acids, each regulatory nucleic acid comprising a nucleotide sequence encoding a single guide RNA (sgRNA) that comprises a targeting sequence that specifically binds to a target sequence within a nucleic acid encoding a target protein of the plurality of target proteins.

In even further embodiments, the kits disclosed herein comprise:

a) a reporter nucleic acid comprising a nucleotide sequence encoding a translation monitor, the translation monitor comprising a reporter nucleotide sequence encoding a reporter protein translated under the control a viral RNA translation element, and
b) a regulatory nucleic acid comprising a nucleotide sequence encoding a single guide RNA (sgRNA) that comprises a targeting sequence that specifically binds to a target sequence within a nucleic acid encoding the target protein.

In additional embodiments, the kits disclosed herein comprise:

a) a reporter nucleic acid comprising a nucleotide sequence encoding a translation monitor, the translation monitor comprising a reporter nucleotide sequence encoding a reporter protein translated under the control of a viral RNA translation element, and
b) a plurality of regulatory nucleic acids, each regulatory nucleic acid comprising a nucleotide sequence encoding a single guide RNA (sgRNA) that comprises a targeting sequence that specifically binds to a target sequence within a nucleic acid encoding a target protein of the plurality of target proteins.

Various details discussed above with respect to the methods of the present disclosure are also applicable to the kits disclosed herein.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method for testing whether a target protein regulates viral RNA translation, the method comprising:

a) introducing into a host cell:

i) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element;

ii) a regulatory nucleic acid comprising a nucleotide sequence encoding a single guide RNA (sgRNA) that comprises a targeting sequence that specifically binds to a target sequence within a nucleic acid encoding the target protein,

wherein the host cell comprises a catalytically inactive CRISPR/Cas effector polypeptide, thereby generating a test host cell; and

b) detecting expression of the first reporter protein and the second reporter protein in the test host cell and in a control host cell, wherein the control host cell comprises the reporter nucleic acid but not the regulatory nucleic acid,

wherein a target protein is considered to regulate translation via the Cap-dependent element if the expression of the first reporter protein in the test host cell is different compared to the expression of the first reporter protein in the control host cell, and

wherein a target protein is considered to regulate translation via the Cap-independent element if the expression of the second reporter protein in the test host cell is different compared to the expression of the second reporter protein in the control host cell.

Aspect 2. The method of Aspect 1, one or both of the Cap-dependent translation element and the Cap-independent translation element is from an RNA virus.

Aspect 3. The method of Aspect 2, wherein the RNA virus is Hepatitis C Virus (HCV), ebolavirus, coronavirus, influenza virus, poliovirus, paramyxovirus, orthomyxovirus, human immunodeficiency virus (HIV) or retrovirus.

Aspect 4. The method of any of preceding Aspects, wherein the first and/or the second reporter protein is green fluorescent protein, red fluorescent protein, luciferase, β-galactosidase, yellow fluorescent protein, blue fluorescent protein, orange fluorescent protein, or a protein that confers antibiotic resistance, and wherein the first reporter protein is different from the second reporter protein.

Aspect 5. The method of any of preceding Aspects, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

Aspect 6. The method of any of preceding Aspects, wherein the regulatory nucleic acid further comprises one or more of: i) a third reporter nucleic acid comprising a nucleotide sequence encoding a third reporter protein; ii) a selectable marker gene; and iii) a promoter driving the expression of the sgRNA.

Aspect 7. The method of any of the preceding Aspects, wherein the reporter nucleic acid and/or the regulatory nucleic acid are in a vector.

Aspect 8. The method of any of the preceding Aspects, wherein the catalytically inactive CRISPR/Cas effector polypeptide is fused to a transcription regulator.

Aspect 9. The method of Aspect 8, wherein the transcription regulator is a transcription inhibitor.

Aspect 10. The method of Aspect 9, wherein the transcription inhibitor is KRAB, Mxil, TUP1, MIG1, CRT1, XTC1, or UME6.

Aspect 11. The method of Aspect 8, wherein the transcription regulator is a transcription activator.

Aspect 12. The method of Aspect 11, wherein the transcription activator is VP64, VP16, VPR, p65AD, or Rta.

Aspect 13. A method of screening a plurality of target proteins for testing whether one or more of the plurality of target proteins regulate viral RNA translation, the method comprising testing the one or more of the plurality of target proteins according to any one the preceding Aspects.

Aspect 14. A method for testing whether a target protein regulates viral RNA translation, the method comprising:

a) introducing into a host cell:

i) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence a second reporter protein translated under the control of a Cap-independent translation element,

ii) a regulatory nucleic acid comprising a nucleotide sequence encoding a single guide RNA (sgRNA) that comprises a targeting sequence that specifically binds to a target sequence within a nucleic acid encoding the target protein,

wherein the host cell comprises a catalytically active CRISPR/Cas effector polypeptide, thereby generating a test host cell; and

b) detecting expression of the first reporter protein and the second reporter protein in the test host cell and in a control host cell, wherein the control host cell comprises the reporter nucleic acid but not the regulatory nucleic acid,

wherein a target protein is considered to regulate translation via the Cap-dependent element if the expression of the first reporter protein in the test host cell is different compared to the expression of the first reporter protein in the control host cell, and

wherein a target protein is considered to regulate translation via the Cap-independent element if the expression of the second reporter protein in the test host cell is different compared to the expression of the second reporter protein in the control host cell.

Aspect 15. The method of Aspect 14, one of the Cap-dependent translation element and the Cap-independent translation element is from an RNA virus.

Aspect 16. The method of Aspect 15, wherein the RNA virus is Hepatitis C Virus (HCV), ebolavirus, coronavirus, influenza virus, poliovirus, paramyxovirus, orthomyxovirus, human immunodeficiency virus (HIV) or retrovirus.

Aspect 17. The method of any of Aspects 14 to 16, wherein the first and/or the second reporter protein is green fluorescent protein, red fluorescent protein, luciferase, β-galactosidase, yellow fluorescent protein, blue fluorescent protein, orange fluorescent protein, or a protein that confers antibiotic resistance, and wherein the first reporter protein is different from the second reporter protein.

Aspect 18. The method of any of Aspects 14 to 17, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

Aspect 19. The method of any of Aspects 14 to 18, wherein the regulatory nucleic acid further comprises one or more of: i) a third reporter nucleic acid comprising a nucleotide sequence encoding a third reporter protein; ii) a selectable marker gene; and iii) a promoter driving the expression of the sgRNA.

Aspect 20. The method of any of Aspects 14 to 19, wherein the reporter nucleic acid and/or the regulatory nucleic acid are in a vector.

Aspect 21. The method of any of Aspects 14 to 20, wherein the targeting sequence of the sgRNA binds within the nucleic acid encoding target protein at a nucleotide sequence that encodes a domain of the target protein.

Aspect 22. A method of screening a plurality of target proteins for testing whether one or more of the plurality of target proteins regulate viral RNA translation, the method comprising testing the one or more of the plurality of target proteins according to any of Aspects 14 to 21.

Aspect 23. A kit for testing whether a target protein regulates viral RNA translation, the kit comprising:

a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and

b) a regulatory nucleic acid comprising a nucleotide sequence encoding a single guide RNA (sgRNA) that comprises a targeting sequence that specifically binds to a target sequence within a nucleic acid encoding the target protein.

Aspect 24. The kit of Aspect 23, further comprising a host cell comprising a catalytically inactive CRISPR/Cas effector polypeptide or a catalytically active CRISPR/Cas effector polypeptide.

Aspect 25. The kit of any of Aspects 23 to 24, one or both of the Cap-dependent translation element and the Cap-independent translation element is from an RNA virus.

Aspect 26. The kit of Aspect 25, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

Aspect 27. The kit of any of Aspects 23 to 26, wherein the first and/or the second reporter protein is green fluorescent protein, red fluorescent protein, luciferase, β-galactosidase, yellow fluorescent protein, blue fluorescent protein, orange fluorescent protein, or a protein that confers antibiotic resistance, and wherein the first reporter protein is different from the second reporter protein.

Aspect 28. The kit of any of Aspects 24 to 27, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

Aspect 29. The kit of any of Aspects 23 to 28, wherein the regulatory nucleic acid further comprises one or more of: i) a third reporter nucleic acid comprising a nucleotide sequence encoding a third reporter protein; ii) a selectable marker gene; and iii) a promoter driving the expression of the

Aspect 30. The kit of any of Aspects 23 to 29, wherein the reporter nucleic acid and/or the regulatory nucleic acid are in a vector.

Aspect 31. The kit of any of Aspects 23 to 30, wherein the targeting sequence of the sgRNA binds within the nucleic acid encoding the target protein at a nucleotide sequence that encodes a domain of the target protein.

Aspect 32. A kit for screening a plurality of target proteins for testing whether one or more of the plurality of target proteins regulate viral RNA translation, the kit comprising:

a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and

b) a plurality of regulatory nucleic acids, each regulatory nucleic acid comprising a nucleotide sequence encoding a single guide RNA (sgRNA) that comprises a targeting sequence that specifically binds to a target sequence within a nucleic acid encoding a target protein of the plurality of target proteins.

Aspect 33. A method for testing whether a target protein regulates viral RNA translation, the method comprising:

a) introducing into a host cell:

i) a reporter nucleic acid comprising a nucleotide sequence encoding a translation monitor, the translation monitor comprising a reporter nucleotide sequence encoding a reporter protein translated under the control of a viral RNA translation element,

ii) a regulatory nucleic acid comprising a nucleotide sequence encoding a single guide RNA (sgRNA) that comprises a targeting sequence that specifically binds to a target sequence within the nucleic acid encoding the target protein,

wherein the host cell comprises a catalytically inactive CRISPR/Cas effector polypeptide, thereby generating a test host cell; and

b) detecting expression of the reporter protein in the test host cell and in a control host cell, wherein the control host cell comprises the reporter nucleic acid but not the regulatory nucleic acid,

wherein a target protein is considered to regulate translation via the viral RNA translation element if the expression of the reporter protein in the test host cell is different compared to the expression of the reporter protein in the control host cell.

Aspect 34. The method of Aspect 33, wherein the viral RNA translation element is from an RNA virus.

Aspect 35. The method of Aspect 34, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

Aspect 36. The method of any of Aspects 33 to 35, wherein the reporter protein is green fluorescent protein, red fluorescent protein, luciferase, β-galactosidase, yellow fluorescent protein, blue fluorescent protein, orange fluorescent protein, or a protein that confers antibiotic resistance.

Aspect 37. The method of any of Aspects 33 to 36, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

Aspect 38. The method of any of Aspects 33 to 37, wherein the regulatory nucleic acid further comprises one or more of: i) a second reporter nucleic acid comprising a nucleotide sequence encoding a second reporter protein, ii) a selectable marker gene, and iii) a promoter driving the expression of the sgRNA.

Aspect 39. The method of any of Aspects 33 to 38, wherein the reporter nucleic acid and/or the regulatory nucleic acid are in a vector.

Aspect 40. The method of any of Aspects 33 to 39, wherein the catalytically inactive CRISPR/Cas effector polypeptide is fused to a transcription regulator.

Aspect 41. The method of Aspect 40, wherein the transcription regulator is a transcription inhibitor.

Aspect 42. The method of Aspect 41, wherein the transcription inhibitor is KRAB, Mxi1, TUP1, MIG1, CRT1, XTC1, or UME6.

Aspect 43. The method of Aspect 40, wherein the transcription regulator is a transcription activator.

Aspect 44. The method of Aspect 43, wherein the transcription activator is VP64, VP16, VPR, p65AD, or Rta.

Aspect 45. A method of screening a plurality of target proteins for testing whether one or more of the plurality of target proteins regulate viral RNA translation, the method comprising testing the one or more of the plurality of target proteins according to any one of Aspects 33 to 44.

Aspect 46. A method for testing whether a target protein regulates viral RNA translation, the method comprising:

a) introducing into a host cell:

i) a reporter nucleic acid comprising a nucleotide sequence encoding a translation monitor, the translation monitor comprising a reporter nucleotide sequence encoding a reporter protein translated under the control a viral RNA translation element,

ii) a regulatory nucleic acid comprising a nucleotide sequence encoding a single guide RNA (sgRNA) that comprises a targeting sequence that specifically binds to a target sequence within a nucleic acid encoding the target protein,

wherein the host cell comprises a catalytically active CRISPR/Cas effector polypeptide, thereby generating a test host cell; and

b) detecting expression of the reporter protein in the test host cell and in a control host cell, wherein the control host cell comprises the reporter nucleic acid but not the regulatory nucleic acid,

wherein a target protein is considered to regulate translation via the viral RNA translation element if the expression of the reporter protein in the test host cell is different compared to the expression of the reporter protein in the control host cell.

Aspect 47. The method of Aspect 46, wherein the viral RNA translation element is from an RNA virus.

Aspect 48. The method of Aspect 47, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

Aspect 49. The method of any of Aspects 46 to 48, wherein the reporter protein is green fluorescent protein, red fluorescent protein, luciferase, β-galactosidase, yellow fluorescent protein, blue fluorescent protein, orange fluorescent protein, or a protein that confers antibiotic resistance.

Aspect 50. The method of any of Aspects 46 to 49, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

Aspect 51. The method of any of Aspects 46 to 50, wherein the regulatory nucleic acid further comprises one or more of: i) a second reporter nucleic acid comprising a nucleotide sequence encoding a second reporter protein, ii) a selectable marker gene, and iii) a promoter driving the expression of the sgRNA.

Aspect 52. The method of any of Aspects 46 to 51, wherein the reporter nucleic acid and/or the regulatory nucleic acid are in a vector.

Aspect 53. The method of any of Aspects 46 to 52, wherein the targeting sequence of the sgRNA binds within the target protein at a nucleotide sequence that encodes a domain of the target protein.

Aspect 54. A method of screening a plurality of target proteins for testing whether one or more of the plurality of target proteins regulate viral RNA translation, the method comprising testing the one or more of the plurality of target proteins according to any of Aspects 46 to 53.

Aspect 55. A kit for testing whether a target protein regulates viral RNA translation, the kit comprising:

a) a reporter nucleic acid comprising a nucleotide sequence encoding a translation monitor, the translation monitor comprising a reporter nucleotide sequence encoding a reporter protein translated under the control a viral RNA translation element, and

b) a regulatory nucleic acid comprising a nucleotide sequence encoding a single guide RNA (sgRNA) that comprises a targeting sequence that specifically binds to a target sequence within a nucleic acid encoding the target protein.

Aspect 56. The kit of Aspect 55, further comprising a host cell comprising catalytically inactive CRISPR/Cas effector polypeptide or a catalytically active CRISPR/Cas effector polypeptide.

Aspect 57. The kit of any of Aspects 55 to 56, wherein the viral RNA translational element is from an RNA virus.

Aspect 58. The kit of Aspect 57, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

Aspect 59. The kit of any of Aspects 55 to 58, wherein the reporter protein is green fluorescent protein, red fluorescent protein, luciferase, β-galactosidase, yellow fluorescent protein, blue fluorescent protein, orange fluorescent protein, or a protein that confers antibiotic resistance.

Aspect 60. The kit of any of Aspects 55 to 59, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

Aspect 61. The kit of any of Aspects 55 to 60, wherein the regulatory nucleic acid further comprises one or more of: i) a second reporter nucleic acid comprising a nucleotide sequence encoding a second reporter protein; ii) a selectable marker gene; and iii) a promoter driving the expression of the sgRNA.

Aspect 62. The kit of any of Aspects 55 to 61, wherein the reporter nucleic acid and/or the regulatory nucleic acid are in a vector.

Aspect 63. The method of any of Aspects 55 to 62, wherein the targeting sequence of the sgRNA binds within the nucleic acid encoding the target protein at a nucleotide sequence that encodes a domain of the target protein.

Aspect 64. A kit for screening a plurality of target proteins for testing whether one or more of the plurality of target proteins regulate viral RNA translation, the kit comprising:

a) a reporter nucleic acid comprising a nucleotide sequence encoding a translation monitor, the translation monitor comprising a reporter nucleotide sequence encoding a reporter protein translated under the control of a viral RNA translation element, and

b) a plurality of regulatory nucleic acids, each regulatory nucleic acid comprising a nucleotide sequence encoding a single guide RNA (sgRNA) that comprises a targeting sequence that specifically binds to a target sequence within a nucleic acid encoding a target protein of the plurality of target proteins.

Aspect 65. The method or kit of any of the preceding Aspects, wherein the Cap-independent translation element is Internal Ribosome Entry Site (IRES) or 5′ untranslated translational regulatory element.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Materials and Methods

Generation of bicistronic reporter cell lines for monitoring cap-dependent and cap-independent RNA translation:

To produce bicistronic reporter cell lines, the liver cancer cell lines HepG2, SNU398, Hep3B, SKHep1, Huh1, Huh7, Alexander were transduced with modified plasmids (Kazadi et al. 2008) expressing bicistronic RFP/GFP reporters for monitoring viral RNA translation. RFP is translated by a cap-dependent cellular or viral RNAs (EF-1 alpha), and GFP is translated by a cap-independent cellular or viral RNAs (HCV, ECMV, MYC, XIAP).

Generation of CRISPRi (CRISPR interference) cell lines and sgRNA libraries for target identification:

To produce CRISPRi cell lines, liver cancer cell lines HepG2, SNU398, Hep3B, SKHep1, Huhl, Huh7, Alexander were transduced with a plasmid (Gilbert et al. 2013) expressing catalytically dead Cas9 fused to a transcriptional repressor peptide KRAB (dCas9-KRAB). The BFP+ cells were then sorted by flow cytometry. Two custom sgRNA CRISPRi libraries focused on total 23 host target genes (two sgRNA per gene) were designed and constructed.

Suppression of target gene by CRISPRi:

Cells expressing dCas9-KRAB were transduced with BFP-linked sgRNAs (Gilbert et al. 2013) targeting candidate genes. The transduced cells were then selected by puromycin for 3 days (day 0) and the BFP+ cells were collected on day 5 for analysis. Total mRNA was isolated using Oligotex mRNA Mini Kit (Qiagen) following manufacturer's instructions. cDNA synthesis and qRT-PCR were performed using SuperScript VILO Master Mix (Thermo Fisher Scientific) and DyNAmo HS SYBR Green qPCR Kits (Thermo Fisher Scientific), respectively. Quantitative PCR analysis was performed on a QuantStudio™ 5 Real-Time PCR System (Thermo Fisher Scientific). All signals were normalized to the levels of β-actin and were quantified using the deltaCt method. Every reaction was performed in triplicate using gene-specific primers.

High-throughput fluorescent assay for translational regulation-mediated by CRISPRi-suppressed target genes:

Cells expressing bicistronic RFP/GFP reporters and dCas9-KRAB were transduced with BFP-linked sgRNAs (Gilbert et al. 2013) targeting candidate genes. The transduced cells were then selected by puromycin for 3 days and the BFP+cells were analyzed in the following weeks. The percentages of GFP^{high} (cap-independent translation), RFP^{high} (cap-dependent translation) and BFP+ (sgRNA expressing) cells were determined by Attune NxT Flow Cytometer (Thermo Fisher Scientific) at different time points. Changes (relative to day 0) were used as readout of the cap-independent and cap-dependent translation activities of cellular and viral RNAs.

Generation of CRISPR cell lines and sgRNA libraries for identification of functional domains within target genes for translation regulation:

To produce CRISPR-Cas9 cell lines, HEK293T cells and liver cancer cells HepG2, were transduced with a plasmid (Shi et al. 2015) expressing catalytically active Cas9. The Cas9+cells were then selected by puromycin. Custom sgRNA CRISPR libraries focused on 4 host target genes (EIF3A, EIF4B, EIF4E and EIFSA) with total 34 sgRNAs targeting domain sequences (EIF3A: 14 sgRNAs; EIF4B: 7 sgRNAs; EIF4E: 9 sgRNAs; EIFSA: 4 sgRNAs) were designed and constructed.

Validation of disruption of domain sequences via T7E1 detection of Cas9-induced cleavage:

Cells expressing Cas9 were transduced with mCherry-linked sgRNAs (Shi et al. 2015) targeting functional domains within candidate genes. Two days after infection, cells were trypsinized, suspended and spun down at 5000g, 4° C. for 3 minutes. Genomic DNA was extracted using the QuickExtract DNA Extraction Solutions (Epicentre), following incubation at 65° C. for 20 minutes and 98° C. for 20 minutes. PCR amplify regions by Q5 DNA polymerase (NEB) and forward and reverse primers designed to amplify regions with mismatches caused by cas9-induced breaks. PCR products were annealed by mixing 200ng DNA with 50 mM KCl, following incubation at 95° C. for 10 minutes and ramp down to 25° C. at the rate of 10° C. per minute. Annealed PCR products were digested with T7 endonuclease I (NEB) in mixture with 10× NEBuffer 2 at 37° C. for 30 minutes. Purple loading dye (NEB) was added to the digested PCR products, and then the mixtures were loaded on a 1.5% TAE agarose gel with SYBR Safe (Thermo Fisher Scientific) at 1:10,000 dilution. The gels were immersed in lx TAE buffer during the run.

Generation of monocistronic reporter cell lines for monitoring cap-independent RNA translation:

To produce monocistronic reporter cell lines, liver cancer cells HepG2 were transduced with modified plasmids expressing hairpin-assisted monocistronic Firefly Luciferase (Kazadi et al. 2008) or monocistronic GFP reporters (Kazadi et al. 2008) for monitoring cap-independent viral RNA translation or cryptic promoter activity, respectively.

### Example 2

### Identification of Host Target Genes For Regulating Viral RNA Translation

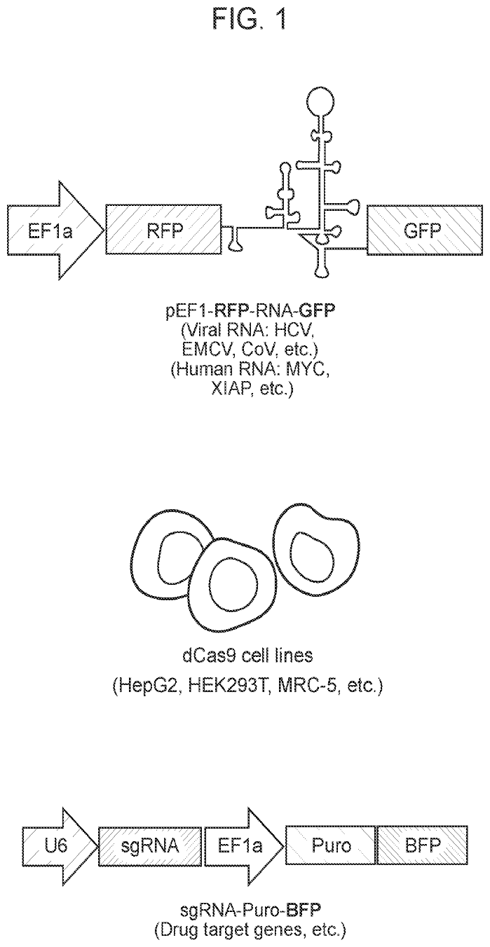

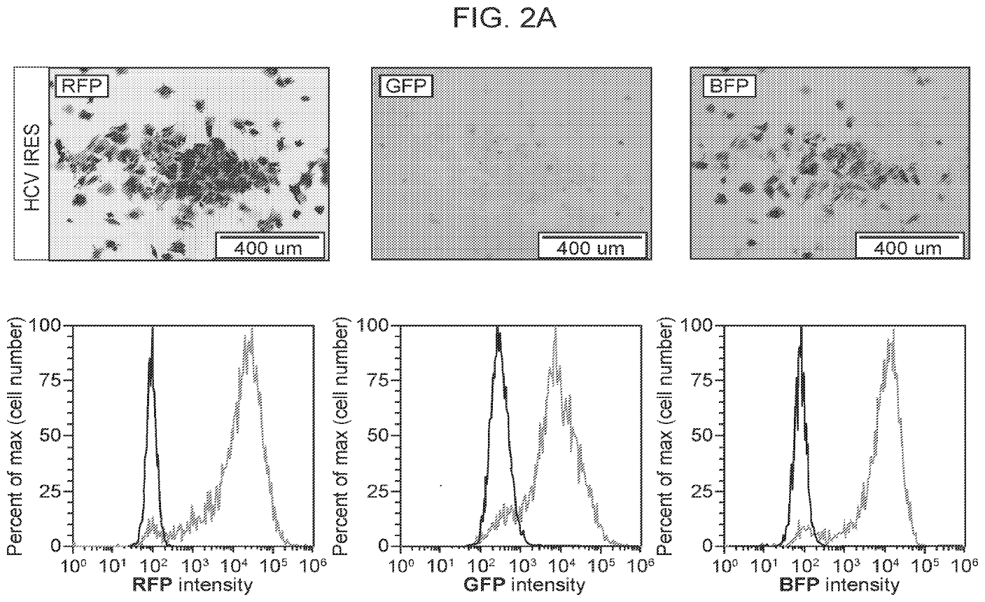

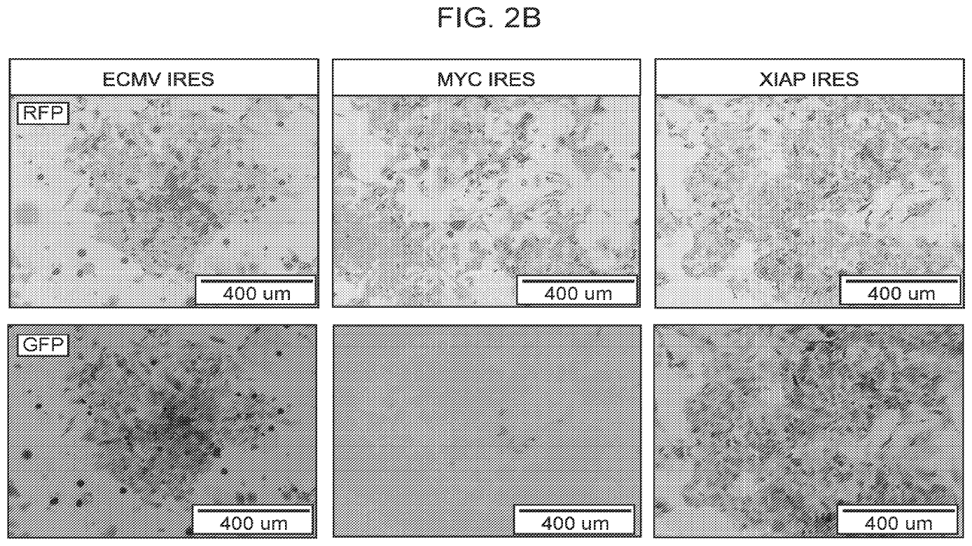

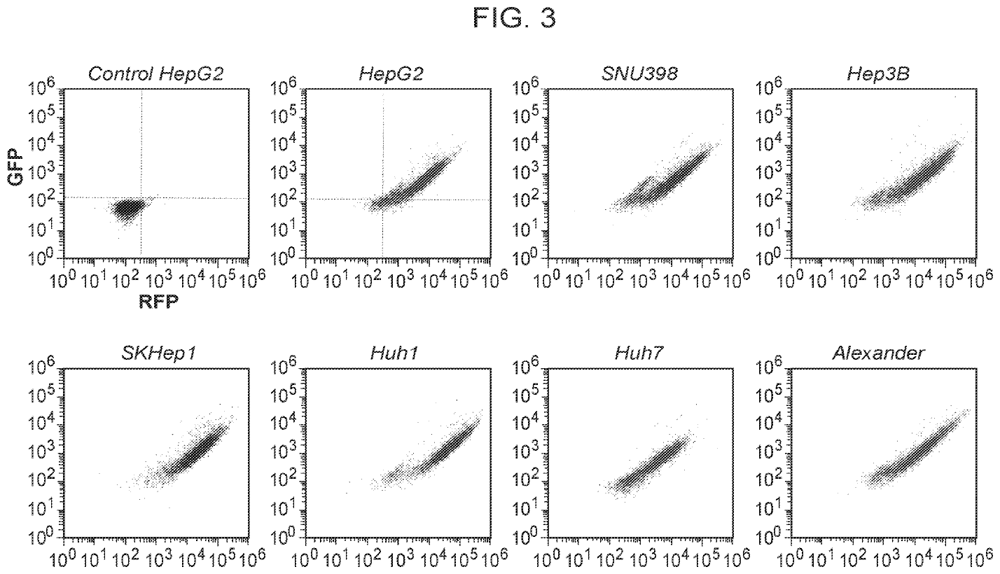

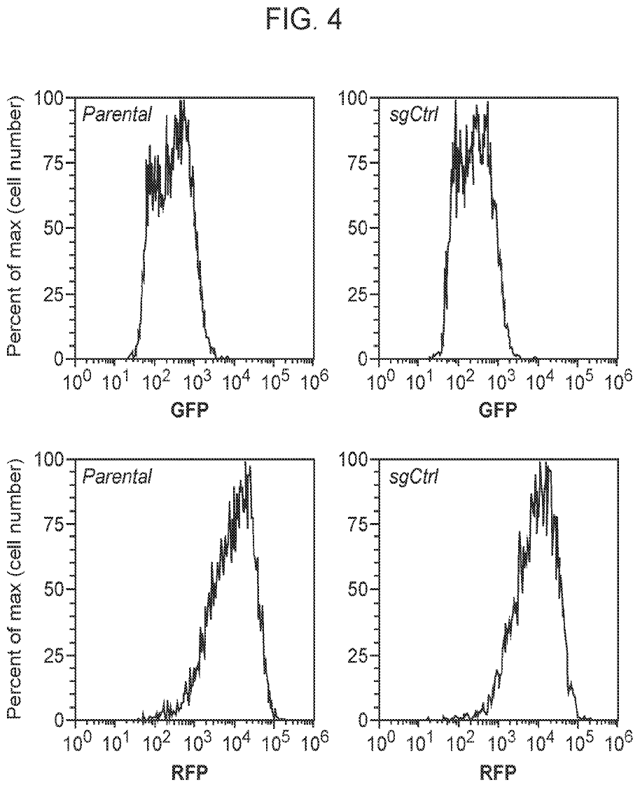

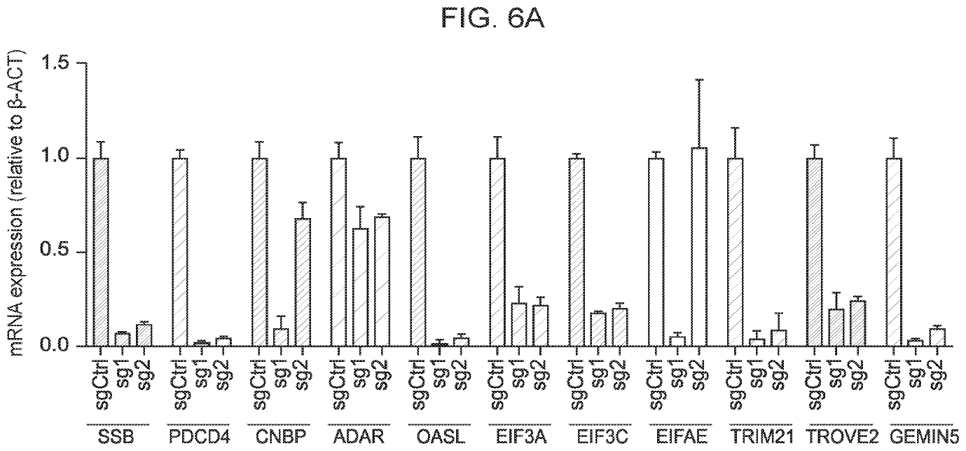

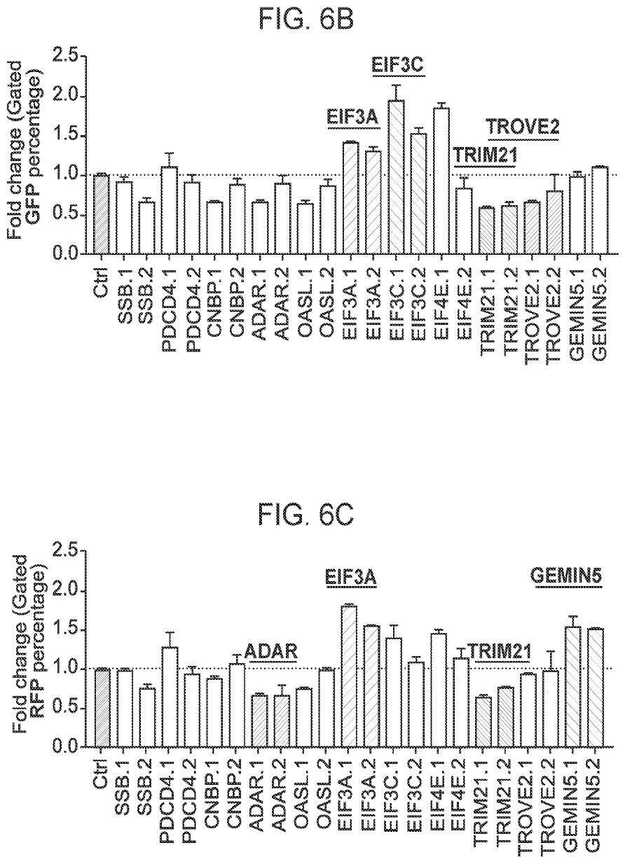

To identify the host genes that are required for the translational regulation of viral RNA, a bicistronic RFP (cap-dependent translation)/GFP (cap-independent translation) reporter system was established in the human liver cancer HepG2 cells expressing a Cas9 or deactivated Cas9 (dCas9) (FIG. 1). The system was validated to monitor both viral and cellular RNAs, including HCV (FIG. 2A), EMCV, MYC, and XIAP (FIG. 2B) IRES RNAs, and was stably expressed across multiple cell lines (FIG. 3). Stably co-expressing a CRISPR system together with the bicistronic RFP/GFP reporter showed no impact on fluorescent output (FIG. 4). 22 sgRNAs that targeting 11 RNA-binding or translation-related genes (2 sgRNAs per gene) were first cloned and introduced into the bicistronic reporter-dCas9 cells (FIG. 5). The CRISPRi-mediated gene suppression of these 11 genes was confirmed by quantitative PCR (qPCR) (FIG. 6A). For cap-independent translation of HCV IRES RNA, silencing translation initiation factors EIF3A and EIF3C increased the percentage of GFP^{high} cells in the HepG2 cell population (FIG. 6B) while silencing autoantigen TRIM21 and TROVE2 decreased the percentage of GFP^{high} cells (FIG. 6B). For cap-dependent translation of human EF-1 alpha, silencing translation initiation factors EIF3A and RNA-binding protein GEMINS significantly increased the percentage of RFP^{high} cells in the HepG2 cell population (FIG. 6C) while silencing RNA-editing protein ADAR and autoantigen TRIM21 significantly decreased the percentage of RFP^{high} cells (FIG. 6C).

### Example 3

### Identification of Host EIF Genes for Regulating Viral RNA Translation

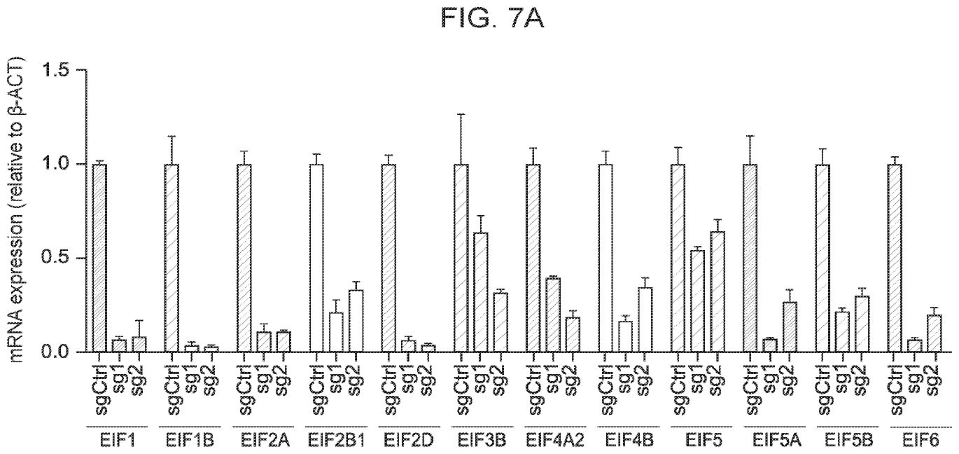

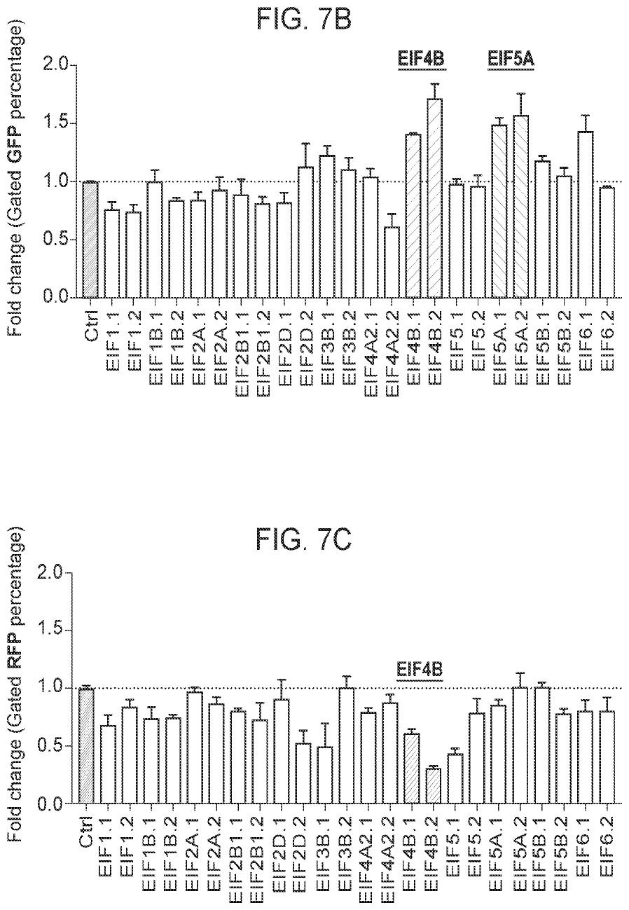

EIF translation initiation factors are varied and execute different functions. To further investigate whether the effect on cap-independent viral translation is only limited to certain EIF genes, the major EIF genes (EIF1-6) (FIG. 5) were screened. 24 sgRNAs that targeting 12 EIF genes (2 sgRNAs per gene) were then cloned and introduced into the bicistronic reporter-dCas9 cells (FIG. 5). The CRISPRi-mediated gene suppression of these 12 genes was confirmed by quantitative PCR (qPCR) (FIG. 7A). For cap-independent translation of HCV IRES RNA, silencing EIF4B and EIF5A significantly increased the percentage of GFP^{high} cells in the HepG2 cell population (FIG. 7B). For cap-dependent translation of human EF-1 alpha, silencing EIF4B significantly decreased the percentage of RFP^{high} cells (FIG. 7C).

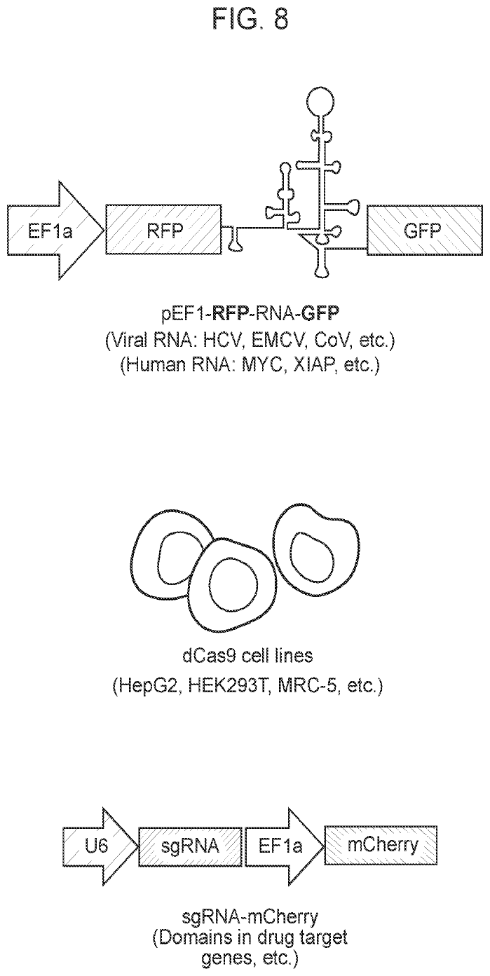

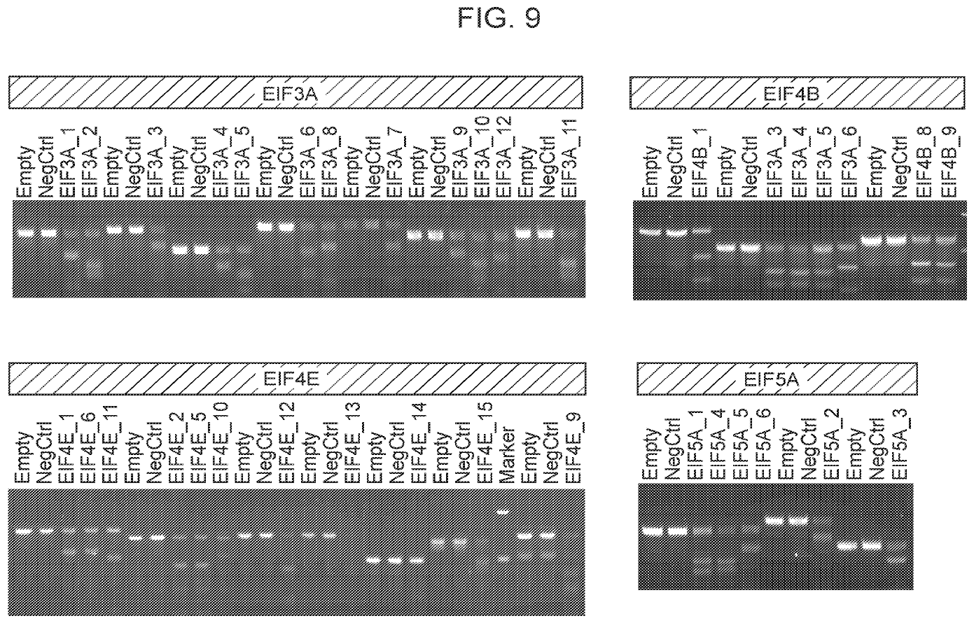

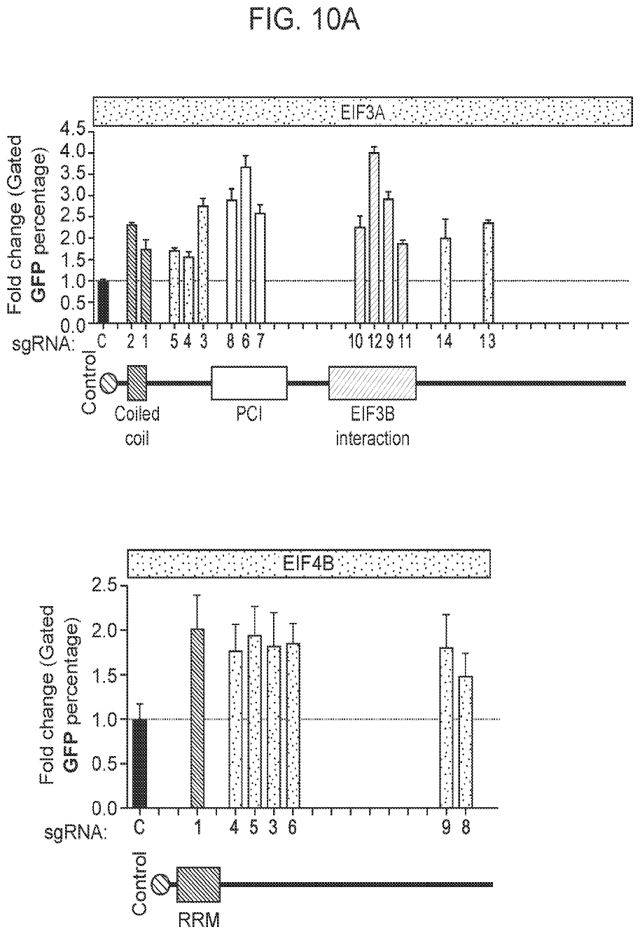

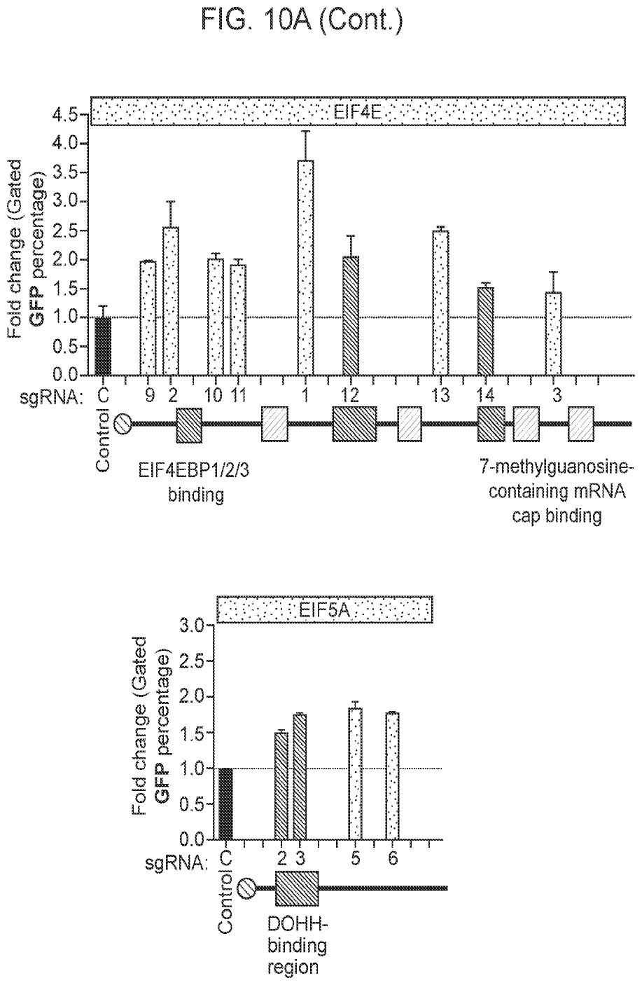

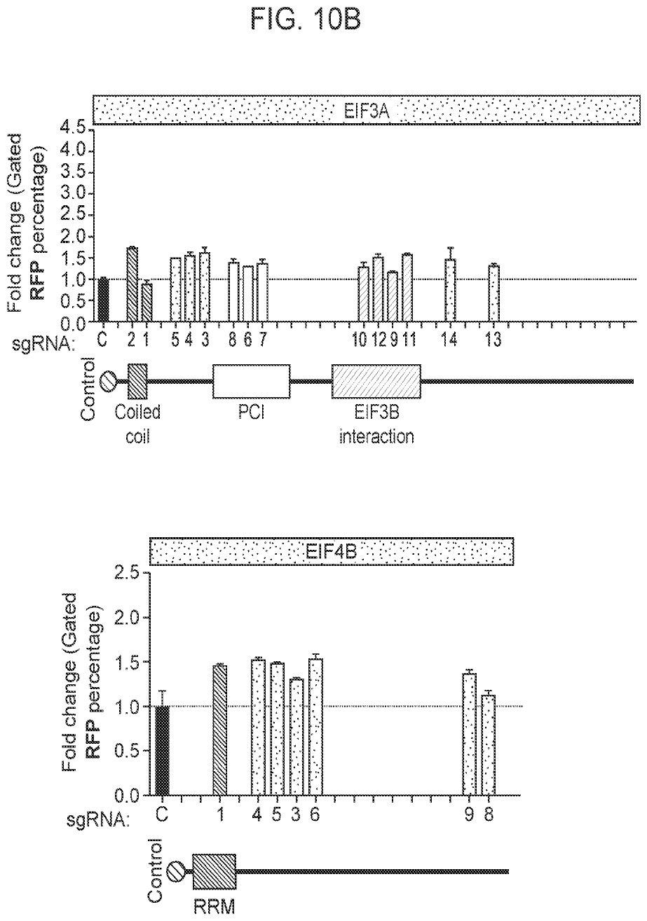

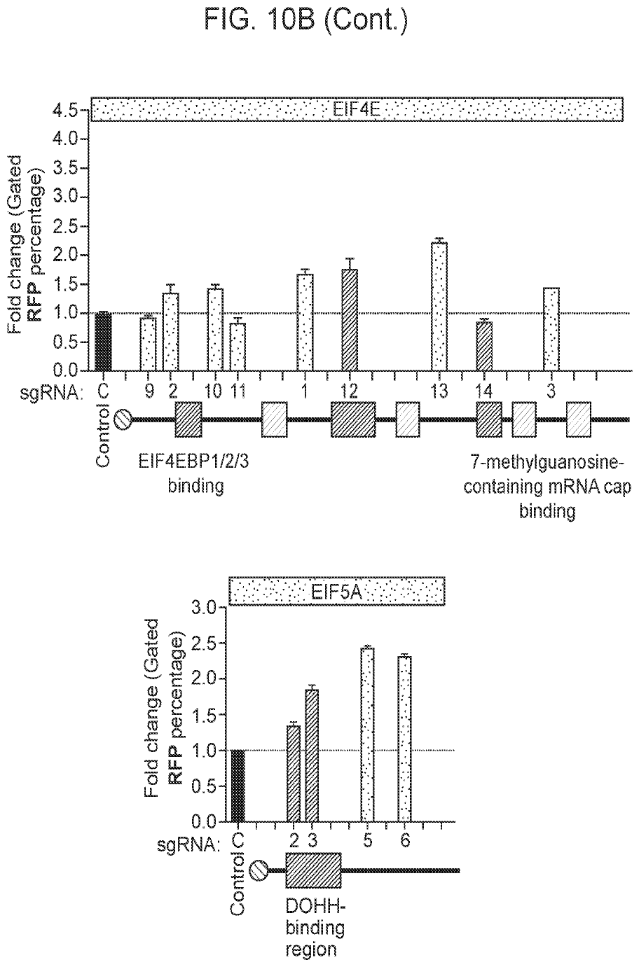

Example 4. Identification of functional domains in host EIF genes for regulating viral RNA translation In order to validate these EIF genes as critical for cap-independent translation of HCV IRES RNA, domains and sequences across the endogenous EIF3A, EIF4B, EIF4E and EIF5A proteins were targeted, using a domain-focused CRISPR-mediated mutagenesis approach (FIG. 8-10). Fourteen, 7, 9, and 4 independent sgRNA for EIF3A, EIF4B, EIF4E and EIF5A, respectively (FIG. 9), were designed and validated. The sgRNAs were expressed in HepG2 liver cells that are engineered to express Cas9 (FIG. 8). After the transduction of sgRNAs, the percentage of GFP^{high} cells significantly increased upon disruption the sequences in EIF3A, especially the PCI and EIF3B-binding domains (FIG. 10). While cells with sgRNAs targeting domains in EIF4B and EIF5A show modest effect (FIG. 10). Interestingly, several sgRNAs targeting novel domains in EIF4E showed a significant increase of the percentage of GFP^{high} cells. These results suggest that some defined and unknown domains in EIF3A and EIF4E plays important roles in HCV IRES translation.

### Example 5

### Validation of the Identified Host-Virus Translational Regulation Using Orthogonal Approaches

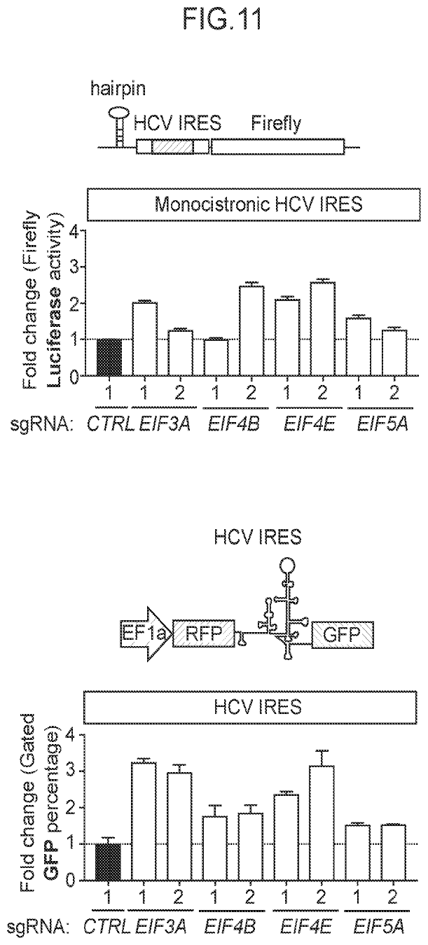

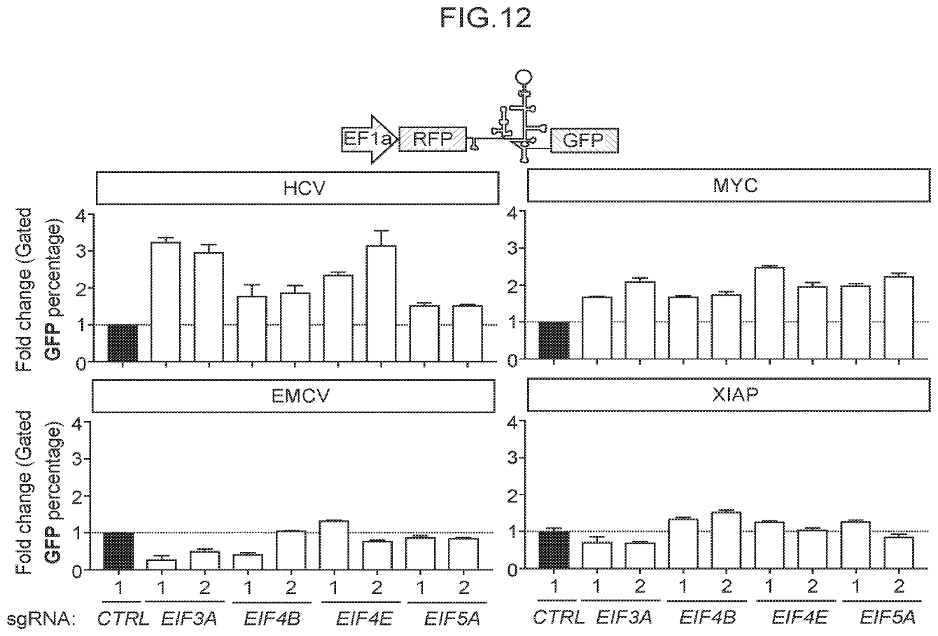

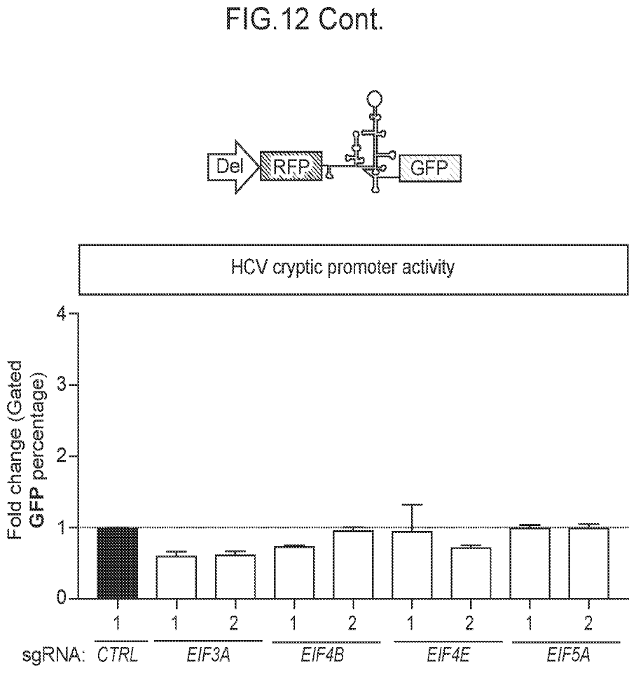

To further validate our findings, the experiment was repeated using a monocistronic report system, in order to rule out the effect derived from the bicistronic vector, and observed the similar result (FIG. 11). The EIF genes-mediated effects were examined on other cellular and viral IRES RNAs. Significant effects on HCV and MYC IRES, but not EMCV and XIAP IRES, were observed (FIG. 12). Moreover, HCV IRES was previously reported to have a cryptic promoter activity. To further examine whether our finding is resulted from HCV IRES's translation or promoter activities, the experiment was repeated using an EF1α promoter-deleted vector (FIG. 12). Suppression of EIF genes did not change GFP signal (FIG. 12), therefore excluding the possibility of effects from the cryptic promoter activity.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for testing whether a target protein regulates viral RNA translation, the method comprising:
a) introducing into a host cell:
i) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element;
ii) a regulatory nucleic acid comprising a nucleotide sequence encoding a single guide RNA (sgRNA) that comprises a targeting sequence that specifically binds to a target sequence within a nucleic acid encoding the target protein, wherein the host cell comprises a catalytically inactive CRISPR/Cas effector polypeptide, thereby generating a test host cell; and
b) detecting expression of the first reporter protein and the second reporter protein in the test host cell and in a control host cell, wherein the control host cell comprises the reporter nucleic acid but not the regulatory nucleic acid,
wherein a target protein is considered to regulate translation via the Cap-dependent element if the expression of the first reporter protein in the test host cell is different compared to the expression of the first reporter protein in the control host cell, and
wherein a target protein is considered to regulate translation via the Cap-independent element if the expression of the second reporter protein in the test host cell is different compared to the expression of the second reporter protein in the control host cell.

2. The method of claim 1, one or both of the Cap-dependent translation element and the Cap-independent translation element is from an RNA virus.

3. The method of claim 2, wherein the RNA virus is Hepatitis C Virus (HCV), ebolavirus, coronavirus, influenza virus, poliovirus, paramyxovirus, orthomyxovirus, human immunodeficiency virus (HIV) or retrovirus.

4. The method of any of preceding claims, wherein the first and/or the second reporter protein is green fluorescent protein, red fluorescent protein, luciferase, β-galactosidase, yellow fluorescent protein, blue fluorescent protein, orange fluorescent protein, or a protein that confers antibiotic resistance, and wherein the first reporter protein is different from the second reporter protein.

5. The method of any of preceding claims, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

6. The method of any of preceding claims, wherein the regulatory nucleic acid further comprises one or more of: i) a third reporter nucleic acid comprising a nucleotide sequence encoding a third reporter protein; ii) a selectable marker gene; and iii) a promoter driving the expression of the sgRNA.

7. The method of any of the preceding claims, wherein the reporter nucleic acid and/or the regulatory nucleic acid are in a vector.

8. The method of any of the preceding claims, wherein the catalytically inactive CRISPR/Cas effector polypeptide is fused to a transcription regulator.

9. The method of claim 8, wherein the transcription regulator is a transcription inhibitor.

10. The method of claim 9, wherein the transcription inhibitor is KRAB, Mxil, TUP1, MIG1, CRT1, XTC1, or UME6.

11. The method of claim 8, wherein the transcription regulator is a transcription activator.

12. The method of claim 11, wherein the transcription activator is VP64, VP16, VPR, p65AD, or Rta.

13. A method of screening a plurality of target proteins for testing whether one or more of the plurality of target proteins regulate viral RNA translation, the method comprising testing the one or more of the plurality of target proteins according to any one the preceding claims.

14. A method for testing whether a target protein regulates viral RNA translation, the method comprising:
a) introducing into a host cell:
i) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence a second reporter protein translated under the control of a Cap-independent translation element,
ii) a regulatory nucleic acid comprising a nucleotide sequence encoding a single guide RNA (sgRNA) that comprises a targeting sequence that specifically binds to a target sequence within a nucleic acid encoding the target protein,
wherein the host cell comprises a catalytically active CRISPR/Cas effector polypeptide, thereby generating a test host cell; and
b) detecting expression of the first reporter protein and the second reporter protein in the test host cell and in a control host cell, wherein the control host cell comprises the reporter nucleic acid but not the regulatory nucleic acid,
wherein a target protein is considered to regulate translation via the Cap-dependent element if the expression of the first reporter protein in the test host cell is different compared to the expression of the first reporter protein in the control host cell, and
wherein a target protein is considered to regulate translation via the Cap-independent element if the expression of the second reporter protein in the test host cell is different compared to the expression of the second reporter protein in the control host cell.

15. The method of claim 14, one of the Cap-dependent translation element and the Cap-independent translation element is from an RNA virus.

16. The method of claim 15, wherein the RNA virus is Hepatitis C Virus (HCV), ebolavirus, coronavirus, influenza virus, poliovirus, paramyxovirus, orthomyxovirus, human immunodeficiency virus (HIV) or retrovirus.

17. The method of any of claims 14 to 16, wherein the first and/or the second reporter protein is green fluorescent protein, red fluorescent protein, luciferase, β-galactosidase, yellow fluorescent protein, blue fluorescent protein, orange fluorescent protein, or a protein that confers antibiotic resistance, and wherein the first reporter protein is different from the second reporter protein.

18. The method of any of claims 14 to 17, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

19. The method of any of claims 14 to 18, wherein the regulatory nucleic acid further comprises one or more of: i) a third reporter nucleic acid comprising a nucleotide sequence encoding a third reporter protein; ii) a selectable marker gene; and iii) a promoter driving the expression of the sgRNA.

20. The method of any of claims 14 to 19, wherein the reporter nucleic acid and/or the regulatory nucleic acid are in a vector.

21. The method of any of claims 14 to 20, wherein the targeting sequence of the sgRNA binds within the nucleic acid encoding target protein at a nucleotide sequence that encodes a domain of the target protein.

22. A method of screening a plurality of target proteins for testing whether one or more of the plurality of target proteins regulate viral RNA translation, the method comprising testing the one or more of the plurality of target proteins according to any of claims 14 to 21.

23. A kit for testing whether a target protein regulates viral RNA translation, the kit comprising:
a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and
b) a regulatory nucleic acid comprising a nucleotide sequence encoding a single guide RNA (sgRNA) that comprises a targeting sequence that specifically binds to a target sequence within a nucleic acid encoding the target protein.

24. The kit of claim 23, further comprising a host cell comprising a catalytically inactive CRISPR/Cas effector polypeptide or a catalytically active CRISPR/Cas effector polypeptide.

25. The kit of any of claims 23 to 24, one or both of the Cap-dependent translation element and the Cap-independent translation element is from an RNA virus.

26. The kit of claim 25, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

27. The kit of any of claims 23 to 26, wherein the first and/or the second reporter protein is green fluorescent protein, red fluorescent protein, luciferase, β-galactosidase, yellow fluorescent protein, blue fluorescent protein, orange fluorescent protein, or a protein that confers antibiotic resistance, and wherein the first reporter protein is different from the second reporter protein.

28. The kit of any of claims 24 to 27, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

29. The kit of any of claims 23 to 28, wherein the regulatory nucleic acid further comprises one or more of: i) a third reporter nucleic acid comprising a nucleotide sequence encoding a third reporter protein; ii) a selectable marker gene; and iii) a promoter driving the expression of the sgRNA.

30. The kit of any of claims 23 to 29, wherein the reporter nucleic acid and/or the regulatory nucleic acid are in a vector.

31. The kit of any of claims 23 to 30, wherein the targeting sequence of the sgRNA binds within the nucleic acid encoding the target protein at a nucleotide sequence that encodes a domain of the target protein.

32. A kit for screening a plurality of target proteins for testing whether one or more of the plurality of target proteins regulate viral RNA translation, the kit comprising:
a) a reporter nucleic acid comprising a nucleotide sequence encoding a bicistronic translation monitor, the bicistronic translation monitor comprising a first reporter nucleotide sequence encoding a first reporter protein translated under the control of a Cap-dependent translation element and a second reporter nucleotide sequence encoding a second reporter protein translated under the control of a Cap-independent translation element, and
b) a plurality of regulatory nucleic acids, each regulatory nucleic acid comprising a nucleotide sequence encoding a single guide RNA (sgRNA) that comprises a targeting sequence that specifically binds to a target sequence within a nucleic acid encoding a target protein of the plurality of target proteins.

33. A method for testing whether a target protein regulates viral RNA translation, the method comprising:
a) introducing into a host cell:
i) a reporter nucleic acid comprising a nucleotide sequence encoding a translation monitor, the translation monitor comprising a reporter nucleotide sequence encoding a reporter protein translated under the control of a viral RNA translation element,
ii) a regulatory nucleic acid comprising a nucleotide sequence encoding a single guide RNA (sgRNA) that comprises a targeting sequence that specifically binds to a target sequence within the nucleic acid encoding the target protein,
wherein the host cell comprises a catalytically inactive CRISPR/Cas effector polypeptide, thereby generating a test host cell; and
b) detecting expression of the reporter protein in the test host cell and in a control host cell, wherein the control host cell comprises the reporter nucleic acid but not the regulatory nucleic acid, wherein a target protein is considered to regulate translation via the viral RNA translation element if the expression of the reporter protein in the test host cell is different compared to the expression of the reporter protein in the control host cell.

34. The method of claim 33, wherein the viral RNA translation element is from an RNA virus.

35. The method of claim 34, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

36. The method of any of claims 33 to 35, wherein the reporter protein is green fluorescent protein, red fluorescent protein, luciferase, β-galactosidase, yellow fluorescent protein, blue fluorescent protein, orange fluorescent protein, or a protein that confers antibiotic resistance.

37. The method of any of claims 33 to 36, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

38. The method of any of claims 33 to 37, wherein the regulatory nucleic acid further comprises one or more of: i) a second reporter nucleic acid comprising a nucleotide sequence encoding a second reporter protein, ii) a selectable marker gene, and iii) a promoter driving the expression of the

39. The method of any of claims 33 to 38, wherein the reporter nucleic acid and/or the regulatory nucleic acid are in a vector.

40. The method of any of claims 33 to 39, wherein the catalytically inactive CRISPR/Cas effector polypeptide is fused to a transcription regulator.

41. The method of claim 40, wherein the transcription regulator is a transcription inhibitor.

42. The method of claim 41, wherein the transcription inhibitor is KRAB, Mxi1, TUP1, MIG1, CRT1, XTC1, or UME6.

43. The method of claim 40, wherein the transcription regulator is a transcription activator.

44. The method of claim 43, wherein the transcription activator is VP64, VP16, VPR, p65AD, or Rta.

45. A method of screening a plurality of target proteins for testing whether one or more of the plurality of target proteins regulate viral RNA translation, the method comprising testing the one or more of the plurality of target proteins according to any one of claims 33 to 44.

46. A method for testing whether a target protein regulates viral RNA translation, the method comprising:
a) introducing into a host cell:
i) a reporter nucleic acid comprising a nucleotide sequence encoding a translation monitor, the translation monitor comprising a reporter nucleotide sequence encoding a reporter protein translated under the control a viral RNA translation element,
ii) a regulatory nucleic acid comprising a nucleotide sequence encoding a single guide RNA (sgRNA) that comprises a targeting sequence that specifically binds to a target sequence within a nucleic acid encoding the target protein,
wherein the host cell comprises a catalytically active CRISPR/Cas effector polypeptide, thereby generating a test host cell; and
b) detecting expression of the reporter protein in the test host cell and in a control host cell, wherein the control host cell comprises the reporter nucleic acid but not the regulatory nucleic acid,
wherein a target protein is considered to regulate translation via the viral RNA translation element if the expression of the reporter protein in the test host cell is different compared to the expression of the reporter protein in the control host cell.

47. The method of claim 46, wherein the viral RNA translation element is from an RNA virus.

48. The method of claim 47, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

49. The method of any of claims 46 to 48, wherein the reporter protein is green fluorescent protein, red fluorescent protein, luciferase, β-galactosidase, yellow fluorescent protein, blue fluorescent protein, orange fluorescent protein, or a protein that confers antibiotic resistance.

50. The method of any of claims 46 to 49, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

51. The method of any of claims 46 to 50, wherein the regulatory nucleic acid further comprises one or more of: i) a second reporter nucleic acid comprising a nucleotide sequence encoding a second reporter protein, ii) a selectable marker gene, and iii) a promoter driving the expression of the sgRNA.

52. The method of any of claims 46 to 51, wherein the reporter nucleic acid and/or the regulatory nucleic acid are in a vector.

53. The method of any of claims 46 to 52, wherein the targeting sequence of the sgRNA binds within the target protein at a nucleotide sequence that encodes a domain of the target protein.

54. A method of screening a plurality of target proteins for testing whether one or more of the plurality of target proteins regulate viral RNA translation, the method comprising testing the one or more of the plurality of target proteins according to any of claims 46 to 53.

55. A kit for testing whether a target protein regulates viral RNA translation, the kit comprising:
a) a reporter nucleic acid comprising a nucleotide sequence encoding a translation monitor, the translation monitor comprising a reporter nucleotide sequence encoding a reporter protein translated under the control a viral RNA translation element, and
b) a regulatory nucleic acid comprising a nucleotide sequence encoding a single guide RNA (sgRNA) that comprises a targeting sequence that specifically binds to a target sequence within a nucleic acid encoding the target protein.

56. The kit of claim 55, further comprising a host cell comprising catalytically inactive CRISPR/Cas effector polypeptide or a catalytically active CRISPR/Cas effector polypeptide.

57. The kit of any of claims 55 to 56, wherein the viral RNA translational element is from an RNA virus.

58. The kit of claim 57, wherein the RNA virus is Hepatitis C Virus (HCV), ebolavirus, coronavirus, influenza virus, poliovirus, paramyxovirus, orthomyxovirus, human immunodeficiency virus (HIV), or retrovirus.

59. The kit of any of claims 55 to 58, wherein the reporter protein is green fluorescent protein, red fluorescent protein, luciferase, β-galactosidase, yellow fluorescent protein, blue fluorescent protein, orange fluorescent protein, or a protein that confers antibiotic resistance.

60. The kit of any of claims 55 to 59, wherein the host cell is 3T3-L1, C2C12, CHO, COS-7, HEK293, HEK293T, HeLa, Hepa1C1c7, Hep G2, Jurkat, MRC-5, NIH-3T3, Raji, or Vero.

61. The kit of any of claims 55 to 60, wherein the regulatory nucleic acid further comprises one or more of: i) a second reporter nucleic acid comprising a nucleotide sequence encoding a second reporter protein; ii) a selectable marker gene; and iii) a promoter driving the expression of the sgRNA.

62. The kit of any of claims 55 to 61, wherein the reporter nucleic acid and/or the regulatory nucleic acid are in a vector.

63. The method of any of claims 55 to 62, wherein the targeting sequence of the sgRNA binds within the nucleic acid encoding the target protein at a nucleotide sequence that encodes a domain of the target protein.

64. A kit for screening a plurality of target proteins for testing whether one or more of the plurality of target proteins regulate viral RNA translation, the kit comprising:
a) a reporter nucleic acid comprising a nucleotide sequence encoding a translation monitor, the translation monitor comprising a reporter nucleotide sequence encoding a reporter protein translated under the control of a viral RNA translation element, and
b) a plurality of regulatory nucleic acids, each regulatory nucleic acid comprising a nucleotide sequence encoding a single guide RNA (sgRNA) that comprises a targeting sequence that specifically binds to a target sequence within a nucleic acid encoding a target protein of the plurality of target proteins.

65. The method or kit of any of the preceding claims, wherein the Cap-independent translation element is Internal Ribosome Entry Site (IRES) or 5′ untranslated translational regulatory element.


SEQUENCE LISTING (123 sequences)

SEQ ID NO: 1 - protein, 113 aa, Homo sapiens
MSAIQNLHSFDPFADASKGDDLLPAGTEDYIHIRIQQRNGRKTLTTVQGIADDYDKKKLVKAFKKKFACNGTVIEHPEYGEVIQLQGDQRKNICQFLVEIGLAKDDQLKVHGF

SEQ ID NO: 2 - protein, 113 aa, Homo sapiens
MSTIQNLQSFDPFADATKGDDLLPAGTEDYIHIRIQQRNGRKTLTTVQGIADDYDKKKLVKAFKKKFACNGTVIEHPEYGEVIQLQGDQRKNICQFLLEVGIVKEEQLKVHGF

SEQ ID NO: 3 - protein, 360 aa, Homo sapiens
MLWNKKATAVLVIASTDVDKTGASYYGEQTLHYIATNGESAVVQLPKNGPIYDVVWNSSSTEFCAVYGFMPAKATIFNLKCDPVFDFGTGPRNAAYYSPHGHILVLAGFGNLRGQMEVWDVKNYKLISKPVASDSTYFAWCPDGEHILTATCAPRLRVNNGYKIWHYTGSILHKYDVPSNAELWQVSWQPFLDGIFPAKTITYQAVPSEVPNEEPKVATAYRPPALRNKPITNSKLHEEEPPQNMKPQSGNDKPLSKTALKNQRKHEAKKAAKQEARSDKSPDLAPTPAPQSTPRNTVSQSISGDPEIDKKIKNLKKKLKAIEQLKEQAATGKQLEKNQLEKIQKETALLQELEDLELGI

SEQ ID NO: 4 - protein, 305 aa, Homo sapiens
MDDKELIEYFKSQMKEDPDMASAVAAIRTLLEFLKRDKGETIQGLRANLTSAIETLCGVDSSVAVSSGGELFLRFISLASLEYSDYSKCKKIMIERGELFLRRISLSRNKIADLCHTFIKDGATILTHAYSRVVLRVLEAAVAAKKRFSVYVTESQPDLSGKKMAKALCHLNVPVTVVLDAAVGYIMEKADLVIVGAEGVVENGGIINKIGTNQMAVCAKAQNKPFYVVAESFKFVRLFPLNQQDVPDKFKYKADTLKVAQTGQDLKEEHPWVDYTAPSLITLLFTDLGVLTPSAVSDELIKLYL

SEQ ID NO: 5 - protein, 584 aa, Homo sapiens
MFAKAFRVKSNTAIKGSDRRKLRADVTTAFPTLGTDQVSELVPGKEELNIVKLYAHKGDAVTVYVSGGNPILFELEKNLYPTVYTLWSYPDLLPTFTTWPLVLEKLVGGADLMLPGLVMPPAGLPQVQKGDLCAISLVGNRAPVAIGVAAMSTAEMLTSGLKGRGFSVLHTYQDHLWRSGNKSSPPSIAPLALDSADLSEEKGSVQMDSTLQGDMRHMTLEGEEENGEVHQAREDKSLSEAPEDTSTRGLNQDSTDSKTLQEQMDELLQQCFLHALKCRVKKADLPLLTSTFLGSHMFSCCPEGRQLDIKKSSYKKLSKFLQQMQQEQIIQVKELSKGVESIVAVDWKHPRITSFVIPEPSPTSQTIQEGSREQPYHPPDIKPLYCVPASMTLLFQESGHKKGSFLEGSEVRTIVINYAKKNDLVDADNKNLVRLDPILCDCILEKNEQHTVMKLPWDSLLTRCLEKLQPAYQVTLPGQEPIVKKGRICPIDITLAQRASNKKVTVVRNLEAYGLDPYSVAAILQQRCQASTTVNPAPGAKDSLQVQIQGNQVHHLGWLLLEEYQLPRKHIQGLEKALKPGKKK

SEQ ID NO: 6 - protein, 1382 aa, Homo sapiens
MPAYFQRPENALKRANEFLEVGKKQPALDVLYDVMKSKKHRTWQKIHEPIMLKYLELCVDLRKSHLAKEGLYQYKNICQQVNIKSLEDVVRAYLKMAEEKTEAAKEESQQMVLDIEDLDNIQTPESVLLSAVSGEDTQDRTDRLLLTPWVKFLWESYRQCLDLLRNNSRVERLYHDIAQQAFKFCLQYTRKAEFRKLCDNLRMHLSQIQRHHNQSTAINLNNPESQSMHLETRLVQLDSAISMELWQEAFKAVEDIHGLFSLSKKPPKPQLMANYYNKVSTVFWKSGNALFHASTLHRLYHLSREMRKNLTQDEMQRMSTRVLLATLSIPITPERTDIARLLDMDGIIVEKQRRLATLLGLQAPPTRIGLINDMVRFNVLQYVVPEVKDLYNWLEVEFNPLKLCERVTKVLNWVREQPEKEPELQQYVPQLQNNTILRLLQQVSQIYQSIEFSRLTSLVPFVDAFQLERAIVDAARHCDLQVRIDHTSRTLSFGSDLNYATREDAPIGPHLQSMPSEQIRNQLTAMSSVLAKALEVIKPAHILQEKEEQHQLAVTAYLKNSRKEHQRILARRQTIEERKERLESLNIQREKEELEQREAELQKVRKAEEERLRQEAKEREKERILQEHEQIKKKTVRERLEQIKKTELGAKAFKDIDIEDLEELDPDFIMAKQVEQLEKEKKELQERLKNQEKKIDYFERAKRLEEIPLIKSAYEEQRIKDMDLWEQQEEERITTMQLEREKALEHKNRMSRMLEDRDLFVMRLKAARQSVYEEKLKQFEERLAEERHNRLEERKRQRKEERRITYYREKEEEEQRRAEEQMLKEREERERAERAKREEELREYQERVKKLEEVERKKRQRELEIEERERRREEERRLGDSSLSRKDSRWGDRDSEGTWRKGPEADSEWRRGPPEKEWRRGEGRDEDRSHRRDEERPRRLGDDEDREPSLRPDDDRVPRRGMDDDRGPRRGPEEDRFSRRGADDDRPSWRNTDDDRPPRRIADEDRGNWRHADDDRPPRRGLDEDRGSWRTADEDRGPRRGMDDDRGPRRGGADDERSSWRNADDDRGPRRGLDDDRGPRRGMDDDRGPRRGMDDDRGPRRGMDDDRGPRRGLDDDRGPWRNADDDRIPRRGAEDDRGPWRNMDDDRLSRRADDDRFPRRGDDSRPGPWRPLVKPGGWREKEKAREESWGPPRESRPSEEREWDREKERDRDNQDREENDKDPERERDRERDVDREDRFRRPRDEGGWRRGPAEESSSWRDSSRRDDRDRDDRRRERDDRRDLRERRDLRDDRDRRGPPLRSEREEVSSWRRADDRKDDRVEERDPPRRVPPPALSRDRERDRDREREGEKEKASWRAEKDRESLRRTKNETDEDGWTTVRR

SEQ ID NO: 7 - protein, 814 aa, Homo sapiens
MQDAENVAVPEAAEERAEPGQQQPAAEPPPAEGLLRPAGPGAPEAAGTEASSEEVGIAEAGPEPEVRTEPAAEAEAASGPSESPSPPAAEELPGSHAEPPVPAQGEAPGEQARDERSDSRAQAVSEDAGGNEGRAAEAEPRALENGDADEPSFSDPEDFVDDVSEEELLGDVLKDRPQEADGIDSVIVVDNVPQVGPDRLEKLKNVIHKIFSKFGKITNDFYPEEDGKTKGYIFLEYASPAHAVDAVKNADGYKLDKQHTFRVNLFTDFDKYMTISDEWDIPEKQPFKDLGNLRYWLEEAECRDQYSVIFESGDRTSIFWNDVKDPVSIEERARWTETYVRWSPKGTYLATFHQRGIALWGGEKFKQIQRFSHQGVQLIDFSPCERYLVTFSPLMDTQDDPQAIIIWDILTGHKKRGFHCESSAHWPIFKWSHDGKFFARMTLDTLSIYETPSMGLLDKKSLKISGIKDFSWSPGGNIIAFWVPEDKDIPARVTLMQLPTRQEIRVRNLFNVVDCKLHWQKNGDYLCVKVDRTPKGTQGVVTNFEIFRMREKQVPVDVVEMKETIIAFAWEPNGSKFAVLHGEAPRISVSFYHVKNNGKIELIKMFDKQQANTIFWSPQGQFVVLAGLRSMNGALAFVDTSDCTVMNIAEHYMASDVEWDPTGRYVVTSVSWWSHKVDNAYWLWTFQGRLLQKNNKDRFCQLLWRPRPPTLLSQEQIKQIKKDLKKYSKIFEQKDRLSQSKASKELVERRRTMMEDFRKYRKMAQELYMEQKNERLELRGGVDTDELDSNVDDWEEETIEFFVTEEIIPLGNQE

SEQ ID NO: 8 - protein, 325 aa, Homo sapiens
MLMSHLQDNIQHADPPVQILYNRTMVQLGICAFRQGLTKDAHNALLDIQSSGRAKELLGQGLLLRSLQERNQEQEKVERRRQVPFHLHINLELLECVYLVSAMLLEIPYMAAHESDARRRMISKQFHHQLRVGERQPLLGPPESMREHVVAASKAMKMGDWKTCHSFIINEKMNGKVWDLFPEADKVRTMLVRKIQEESLRTYLFTYSSVYDSISMETLSDMFELDLPTVHSIISKMIINEELMASLDQPTQTVVMHRTEPTAQQNLALQLAEKLGSLVENNERVFDHKQGTYGGYFRDQKDGYRKNEGYMRRGGYRQQQSQTAY

SEQ ID NO: 9 - protein, 179 aa, Homo sapiens
MRDPIRILVKKEELTLEGIKQFYINVEREEWKLDTLCDLYETLTITQAVIFLNTRRKVDWLTEKMHARDFTVSALHGDMDQKERDVIMREFRSGSSRVLITTDLLARGIDVQQVSLVINYDLPTNRENYIHRIGRGGRFGRKGVAINFVTEEDKRILRDIETFYNTTVEEMPMNVADLI

SEQ ID NO: 10 - protein, 611 aa, Homo sapiens
MAASAKKKNKKGKTISLTDFLAEDGGTGGGSTYVSKPVSWADETDDLEGDVSTTWHSNDDDVYRAPPIDRSILPTAPRAAREPNIDRSRLPKSPPYTAFLGNLPYDVTEESIKEFFRGLNISAVRLPREPSNPERLKGFGYAEFEDLDSLLSALSLNEESLGNRRIRVDVADQAQDKDRDDRSFGRDRNRDSDKTDTDWRARPATDSFDDYPPRRGDDSFGDKYRDRYDSDRYRDGYRDGYRDGPRRDMDRYGGRDRYDDRGSRDYDRGYDSRIGSGRRAFGSGYRRDDDYRGGGDRYEDRYDRRDDRSWSSRDDYSRDDYRRDDRGPPQRPKLNLKPRSTPKEDDSSASTSQSTRAASIFGGAKPVDTAAREREVEERLQKEQEKLQRQLDEPKLERRPRERHPSWRSEETQERERSRTGSESSQTGTSTTSSRNARRRESEKSLENETLNKEEDCHSPTSKPPKPDQPLKVMPAPPPKENAWVKRSSNPPARSQSSDTEQQSPTSGGGKVAPAQPSEEGPGRKDENKVDGMNAPKGQTGNSSRGPGDGGNRDHWKESDRKDGKKDQDSRSAPEPKKPEENPASKFSSASKYAALSVDGEDENEGEDYAD

SEQ ID NO: 11 - protein, 248 aa, Homo sapiens
MATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETRWDLAMLPRLVSNFWPQVILPLQPPKVLELQLLCLIGESFDDYSDDVCGAVVNVRAKGDKIAIWTTECENREAVTHIGRVYKERLGLPPKIVIGYQSHADTATKSGSTTKNRFVV

SEQ ID NO: 12 - protein, 431 aa, Homo sapiens [sequence omitted]

SEQ ID NO: 13 - protein, 154 aa, Homo sapiens
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 14 - protein, 1220 aa, Homo sapiens
MGKKQKNKSEDSTKDDIDLDALAAEIEGAGAAKEQEPQKSKGKKKKEKKKQDFDEDDILKELEELSLEAQGIKADRETVAVKPTENNEEEFTSKDKKKKGQKGKKQSFDDNDSEELEDKDSKSKKTAKPKVEMYSGSDDDDDFNKLPKKAKGKAQKSNKKWDGSEEDEDNSKKIKERSRINSSGESGDESDEFLQSRKGQKKNQKNKPGPNIESGNEDDDASFKIKTVAQKKAEKKERERKKRDEEKAKLRKLKEKEELETGKKDQSKQKESQRKFEEETVKSKVTVDTGVIPASEEKAETPTAAEDDNEGDKKKKDKKKKKGEKEEKEKEKKKGPSKATVKAMQEALAKLKEEEERQKREEEERIKRLEELEAKRKEEERLEQEKRERKKQKEKERKERLKKEGKLLTKSQREARARAEATLKLLQAQGVEVPSKDSLPKKRPIYEDKKRKKIPQQLESKEVSESMELCAAVEVMEQGVPEKEETPPPVEPEEEEDTEDAGLDDWEAMASDEETEKVEGNKVHIEVKENPEEEEEEEEEEEEDEESEEEEEEEGESEGSEGDEEDEKVSDEKDSGKTLDKKPSKEMSSDSEYDSDDDRTKEERAYDKAKRRIEKRRLEHSKNVNTEKLRAPIICVLGHVDTGKTKILDKLRHTHVQDGEAGGITQQIGATNVPLEAINEQTKMIKNFDRENVRIPGMLIIDTPGHESFSNLRNRGSSLCDIAILVVDIMHGLEPQTIESINLLKSKKCPFIVALNKIDRLYDWKKSPDSDVAATLKKQKKNTKDEFEERAKAIIVEFAQQGLNAALFYENKDPRTFVSLVPTSAHTGDGMGSLIYLLVELTQTMLSKRLAHCEELRAQVMEVKALPGMGTTIDVILINGRLKEGDTIIVPGVEGPIVTQIRGLLLPPPMKELRVKNQYEKHKEVEAAQGVKILGKDLEKTLAGLPLLVAYKEDEIPVLKDELIHELKQTLNAIKLEEKGVYVQASTLGSLEALLEFLKTSEVPYAGINIGPVHKKDVMKASVMLEHDPQYAVILAFDVRIERDAQEMADSLGVRIFSAEIIYHLFDAFTKYRQDYKKQKQEEFKHIAVFPCKIKILPQYIFNSRDPIVMGVTVEAGQVKQGTPMCVPSKNFVDIGIVTSIEINHKQVDVAKKGQEVCVKIEPIPGESPKMFGRHFEATDILVSKISRQSIDALKDWFRDEMQKSDWQLIVELKKVFEII

SEQ ID NO: 15 - protein, 245 aa, Homo sapiens
MAVRASFENNCEIGCFAKLTNTYCLVAIGGSENFYSVFEGELSDTIPVVHASIAGCRIIGRMCVGNRHGLLVPNNTTDQELQHIRNSLPDTVQIRRVEERLSALGNVTTCNDYVALVHPDLDRETEEILADVLKVEVFRQTVADQVLVGSYCVFSNQGGLVHPKTSIEDQDELSSLLQVPLVAGTVNRGSEVIAAGMVVNDWCAFCGLDTTSTELSVVESVFKLNEAQPSTIATSMRDSLIDSLT

SEQ ID NO: 16 - protein, 469 aa, Homo sapiens
MDVENEQILNVNPADPDNLSDSLFSGDEENAGTEEIKNEINGNWISASSINEARINAKAKRRLRKNSSRDSGRGDSVSDSGSDALRSGLTVPTSPKGRLLDRRSRSGKGRGLPKKGGAGGKGVWGTPGQVYDVEEVDVKDPNYDDDQENCVYETVVLPLDERAFEKTLTPIIQEYFEHGDTNEVAEMLRDLNLGEMKSGVPVLAVSLALEGKASHREMTSKLLSDLCGTVMSTTDVEKSFDKLLKDLPELALDTPRAPQLVGQFIARAVGDGILCNTYIDSYKGTVDCVQARAALDKATVLLSMSKGGKRKDSVWGSGGGQQSVNHLVKEIDMLLKEYLLSGDISEAEHCLKELEVPHFHHELVYEAIIMVLESTGESTFKMILDLLKSLWKSSTITVDQMKRGYERIYNEIPDINLDVPHSYSVLERFVEECFQAGIISKQLRDLCPSRGRKRFVSEGDGGRLKPESY

SEQ ID NO: 17 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 18 - protein, 514 aa, Homo sapiens
MALMQELYSTPASRLDSFVAQWLQPHREWKEEVLDAVRTVEEFLRQEHFQGKRGLDQDVRVLKVVKVGSFGNGTVLRSTREVELVAFLSCFHSFQEAAKHHKDVLRLIWKTMWQSQDLLDLGLEDLRMEQRVPDALVFTIQTRGTAEPITVTIVPAYRALGPSLPNSQPPPEVYVSLIKACGGPGNFCPSFSELQRNFVKHRPTKLKSLLRLVKHWYQQYVKARSPRANLPPLYALELLTIYAWEMGTEEDENFMLDEGFTTVMDLLLEYEVICIYWTKYYTLHNAIIEDCVRKQLKKERPIILDPADPTLNVAEGYRWDIVAQRASQCLKQDCCYDNRENPISSWNVKRARDIHLTVEQRGYPDFNLIVNPYEPIRKVKEKIRRTRGYSGLQRLSFQVPGSERQLLSSRCSLAKYGIFSHTHIYLLETIPSEIQVFVKNPDGGSYAYAINPNSFILGLKQQIEDQQGLPKKQQQLEFQGQVLQDWLGLGIYGIQDSDTLILSKKKGEALFPAS

SEQ ID NO: 19 - protein, 1507 aa, Homo sapiens [sequence omitted]

SEQ ID NO: 20 - protein, 170 aa, Homo sapiens
MSSNECFKCGRSGHWARECPTGGGRGRGMRSRGRGFQFVSSSLPDICYRCGESGHLAKDCDLQEDACYNCGRGGHIAKDCKEPKREREQCCYNCGKPGHLARDCDHADEQKCYSCGEFGHIQKDCTKVKCYRCGETGHVAINCSKTSEVNCYRCGESGHLARECTIEATA

SEQ ID NO: 21 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 22 - protein, 475 aa, Homo sapiens [sequence omitted]

SEQ ID NO: 23 - protein, 525 aa, Homo sapiens
MEESVNQMQPLNEKQIANSQDGYVWQVTDMNRLHRFLCFGSEGGTYYIKEQKLGLENAEALIRLIEDGRGCEVIQEIKSFSQEGRTTKQEPMLFALAICSQCSDISTKQAAFKAVSEVCRIPTHLFTFIQFKKDLKESMKCGMWGRALRKAIADWYNEKGGMALALAVTKYKQRNGWSHKDLLRLSHLKPSSEGLAIVTKYITKGWKEVHELYKEKALSVETEKLLKYLEAVEKVKRTRDELEVIHLIEEHRLVREHLLTNHLKSKEVWKALLQEMPLTALLRNLGKMTANSVLEPGNSEVSLVCEKLCNEKLLKKARIHPFHILIALETYKTGHGLRGKLKWRPDEEILKALDAAFYKTFKTVEPTGKRFLLAVDVSASMNQRVLGSILNASTVAAAMCMVVTRTEKDSYVVAFSDEMVPCPVTTDMTLQQVLMAMSQIPAGGTDCSLPMIWAQKTNTPADVFIVFTDNETFAGGVHPAIALREYRKKMDIPAKLIVCGMTSNGFTIADPDDRALQNTLLNKSF

SEQ ID NO: 24 - protein, 1368 aa, Streptococcus pyogenes [sequence omitted]

SEQ ID NO: 25 - protein, 1368 aa, Artificial sequence
  synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - protein, 1368 aa, Artificial sequence
  synthetic sequence
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 27 - protein, 1368 aa, Artificial sequence
  synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 28 - protein, 1368 aa, Artificial sequence
  synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1367 aa, Artificial sequence
  synthetic sequence
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 31 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 32 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 33 - protein, 1306 aa, Artificial sequence
  synthetic sequence
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 34 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 35 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 36 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 37 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 38 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 39 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 40 - DNA, 368 bp, Hepatitis C Virus
gccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaga

SEQ ID NO: 41 - DNA, 33 bp, Artificial sequence
  synthetic sequence
ttggcggtgagactagaaggcgggtttaagagc

SEQ ID NO: 42 - DNA, 33 bp, Artificial sequence
  synthetic sequence
ttggcttcggtgagactagaagggtttaagagc

SEQ ID NO: 43 - DNA, 33 bp, Artificial sequence
  synthetic sequence
ttggggccagagactctccgagggtttaagagc

SEQ ID NO: 44 - DNA, 33 bp, Artificial sequence
  synthetic sequence
ttgggccggctgaccaggaacctgtttaagagc

SEQ ID NO: 45 - DNA, 33 bp, Artificial sequence
  synthetic sequence
ttggagaggcgcaggccaaagtggtttaagagc

SEQ ID NO: 46 - DNA, 33 bp, Artificial sequence
  synthetic sequence
ttggcccgcgtgtggcgcaggcagtttaagagc

SEQ ID NO: 47 - DNA, 33 bp, Artificial sequence
  synthetic sequence
ttggctgcgcgccgggcccaagagtttaagagc

SEQ ID NO: 48 - DNA, 33 bp, Artificial sequence
  synthetic sequence
ttggcgcactggaagtggccccggtttaagagc

SEQ ID NO: 49 - DNA, 33 bp, Artificial sequence
  synthetic sequence
ttggggtactctcgggacagagagtttaagagc

SEQ ID NO: 50 - DNA, 33 bp, Artificial sequence
  synthetic sequence
ttgggcccagcagcggtactctcgtttaagagc

SEQ ID NO: 51 - DNA, 33 bp, Artificial sequence
  synthetic sequence
ttggacgaaggggaaccagcgtagtttaagagc

SEQ ID NO: 52 - DNA, 33 bp, Artificial sequence
  synthetic sequence
ttggggcgcgtgggaccttacgcgtttaagagc

SEQ ID NO: 53 - DNA, 33 bp, Artificial sequence
  synthetic sequence
ttggcggctatcacagccctctagtttaagagc

SEQ ID NO: 54 - DNA, 33 bp, Artificial sequence
  synthetic sequence
ttggtagccggtagccacccaccgtttaagagc

SEQ ID NO: 55 - DNA, 33 bp, Artificial sequence
  synthetic sequence
ttggggccaaaggcaatactcacgtttaagagc

SEQ ID NO: 56 - DNA, 33 bp, Artificial sequence
  synthetic sequence
ttggtgccaccggctggacgagggtttaagagc

SEQ ID NO: 57 - DNA, 33 bp, Artificial sequence
  synthetic sequence
ttggcagcctagccaggagggtagtttaagagc

SEQ ID NO: 58 - DNA, 33 bp, Artificial sequence
  synthetic sequence
ttggggttcactcacctttacaggtttaagagc

SEQ ID NO: 59 - DNA, 33 bp, Artificial sequence
  synthetic sequence
ttgggaaccgaacctggaatcccgtttaagagc

SEQ ID NO: 60 - DNA, 33 bp, Artificial sequence
  synthetic sequence
ttggcgacaagcaggagcggtccgtttaagagc

SEQ ID NO: 61 - DNA, 33 bp, Artificial sequence
  synthetic sequence
ttgggcgtgaggctacgagcggtgtttaagagc

SEQ ID NO: 62 - DNA, 33 bp, Artificial sequence
  synthetic sequence
ttggtagcctcacgccttaggtagtttaagagc

SEQ ID NO: 63 - DNA, 33 bp, Artificial sequence
  synthetic sequence
ttggagacgccgggcccgcccaagtttaagagc

SEQ ID NO: 64 - DNA, 33 bp, Artificial sequence
  synthetic sequence
ttggggaacgtcgggaagcgagggtttaagagc

SEQ ID NO: 65 - DNA, 33 bp, Artificial sequence
  synthetic sequence
ttggggcgagggcgcgagagaaggtttaagagc

SEQ ID NO: 66 - DNA, 33 bp, Artificial sequence
  synthetic sequence
ttggaggcaggaagaggcgaagggtttaagagc

SEQ ID NO: 67 - DNA, 33 bp, Artificial sequence
  synthetic sequence
ttggcgtccacgccgctcttgacgtttaagagc

SEQ ID NO: 68 - DNA, 33 bp, Artificial sequence
  synthetic sequence
ttgggaatacgaaagcccagagggtttaagagc

SEQ ID NO: 69 - DNA, 33 bp, Artificial sequence
  synthetic sequence
ttggtgtcagactggctcgcagggtttaagagc

SEQ ID NO: 70 - DNA, 33 bp, Artificial sequence
  synthetic sequence
ttgggtgcgccggaagaggggtagtttaagagc

SEQ ID NO: 71 - DNA, 33 bp, Artificial sequence
  synthetic sequence
ttgggccaggccctcagccgtgggtttaagagc

SEQ ID NO: 72 - DNA, 33 bp, Artificial sequence
  synthetic sequence
ttgggggaatgtcaagaaagcgagtttaagagc

SEQ ID NO: 73 - DNA, 33 bp, Artificial sequence
  synthetic sequence
ttggccaacgctgcctactcgcagtttaagagc

SEQ ID NO: 74 - DNA, 33 bp, Artificial sequence
  synthetic sequence
ttgggcctactcgcagggctccggtttaagagc

SEQ ID NO: 75 - DNA, 33 bp, Artificial sequence
  synthetic sequence
ttgggcatacctgttataatccggtttaagagc

SEQ ID NO: 76 - DNA, 33 bp, Artificial sequence
  synthetic sequence
ttggtggctccgcggattataacgtttaagagc

SEQ ID NO: 77 - DNA, 33 bp, Artificial sequence
  synthetic sequence
ttggatagcgggcgcgtcgagctgtttaagagc

SEQ ID NO: 78 - DNA, 33 bp, Artificial sequence
  synthetic sequence
ttggccgagcgcggccaaggactgtttaagagc

SEQ ID NO: 79 - DNA, 33 bp, Artificial sequence
  synthetic sequence
ttggcagagcgagaacattccaggtttaagagc

SEQ ID NO: 80 - DNA, 33 bp, Artificial sequence
  synthetic sequence
ttggccttcaccggactcacctcgtttaagagc

SEQ ID NO: 81 - DNA, 33 bp, Artificial sequence
  synthetic sequence
ttgggctgcttggttggtcagtggtttaagagc

SEQ ID NO: 82 - DNA, 33 bp, Artificial sequence
  synthetic sequence
ttggccgccaggcggccacgccggtttaagagc

SEQ ID NO: 83 - DNA, 33 bp, Artificial sequence
  synthetic sequence
ttggaggggttgggtccgtacgggtttaagagc

SEQ ID NO: 84 - DNA, 33 bp, Artificial sequence
  synthetic sequence
ttggggcggccgccgtggctcaggtttaagagc

SEQ ID NO: 85 - DNA, 33 bp, Artificial sequence
  synthetic sequence
ttggcgcgtgcggagcttgttacgtttaagagc

SEQ ID NO: 86 - DNA, 33 bp, Artificial sequence
  synthetic sequence
ttgggcgggacccgaggacggaggtttaagagc

SEQ ID NO: 87 - DNA, 25 bp, Artificial sequence
  synthetic sequence
caccggcagatggtcttagatatag

SEQ ID NO: 88 - DNA, 25 bp, Artificial sequence
  synthetic sequence
caccgaatctctggaggatgttgtt

SEQ ID NO: 89 - DNA, 25 bp, Artificial sequence
  synthetic sequence
caccgctgcctccaatacacgcgta

SEQ ID NO: 90 - DNA, 25 bp, Artificial sequence
  synthetic sequence
caccggctgggcaatatcatggtac

SEQ ID NO: 91 - DNA, 25 bp, Artificial sequence
  synthetic sequence
caccgactgcctgtaagactcccac

SEQ ID NO: 92 - DNA, 25 bp, Artificial sequence
  synthetic sequence
caccgactctgtgagcgagtcacaa

SEQ ID NO: 93 - DNA, 25 bp, Artificial sequence
  synthetic sequence
caccgggcacatactgctgcaattc

SEQ ID NO: 94 - DNA, 25 bp, Artificial sequence
  synthetic sequence
caccgctacttcaagccaattgtaa

SEQ ID NO: 95 - DNA, 25 bp, Artificial sequence
  synthetic sequence
caccgtttgataaagagcgcttacg

SEQ ID NO: 96 - DNA, 25 bp, Artificial sequence
  synthetic sequence
caccgggctaaacaggttgaacaac

SEQ ID NO: 97 - DNA, 25 bp, Artificial sequence
  synthetic sequence
caccgaatgcagctagaacgtgaaa

SEQ ID NO: 98 - DNA, 25 bp, Artificial sequence
  synthetic sequence
caccgttttgaaagagccaaacgtt

SEQ ID NO: 99 - DNA, 25 bp, Artificial sequence
  synthetic sequence
caccgagaacgagaacggcgtagag

SEQ ID NO: 100 - DNA, 25 bp, Artificial sequence
  synthetic sequence
caccgggaagagctacgagagtatc

SEQ ID NO: 101 - DNA, 25 bp, Artificial sequence
  synthetic sequence
caccggtcatcgttactgtgccaag

SEQ ID NO: 102 - DNA, 25 bp, Artificial sequence
  synthetic sequence
caccgcccgatacccatcccgatac

SEQ ID NO: 103 - DNA, 25 bp, Artificial sequence
  synthetic sequence
caccggttatgattcagaccggtat

SEQ ID NO: 104 - DNA, 25 bp, Artificial sequence
  synthetic sequence
caccgattcagaccggtatcgggat

SEQ ID NO: 105 - DNA, 25 bp, Artificial sequence
  synthetic sequence
caccggtatcgggatggcccacgcc

SEQ ID NO: 106 - DNA, 25 bp, Artificial sequence
  synthetic sequence
caccggttcccagtttggccttttg

SEQ ID NO: 107 - DNA, 25 bp, Artificial sequence
  synthetic sequence
caccggatgggatgaatgccccaaa

SEQ ID NO: 108 - DNA, 25 bp, Artificial sequence
  synthetic sequence
caccgcaaacttggagatcagccgc

SEQ ID NO: 109 - DNA, 25 bp, Artificial sequence
  synthetic sequence
caccgtaatcccccgactacagaag

SEQ ID NO: 110 - DNA, 25 bp, Artificial sequence
  synthetic sequence
caccgaagctgttacacatataggg

SEQ ID NO: 111 - DNA, 25 bp, Artificial sequence
  synthetic sequence
caccgtaagatggcgactgtcgaac

SEQ ID NO: 112 - DNA, 25 bp, Artificial sequence
  synthetic sequence
caccgttaaacatcccctacagaac

SEQ ID NO: 113 - DNA, 25 bp, Artificial sequence
  synthetic sequence
caccgttatttttagatgggcactc

SEQ ID NO: 114 - DNA, 25 bp, Artificial sequence
  synthetic sequence
caccgtggatatggttgtacagact

SEQ ID NO: 115 - DNA, 25 bp, Artificial sequence
  synthetic sequence
caccggcttttggctagagacagta

SEQ ID NO: 116 - DNA, 25 bp, Artificial sequence
  synthetic sequence
caccgtttaagcttctgtgccttat

SEQ ID NO: 117 - DNA, 25 bp, Artificial sequence
  synthetic sequence
caccgctaggagggtatacaaggaa

SEQ ID NO: 118 - DNA, 25 bp, Artificial sequence
  synthetic sequence
caccgggacagcggggaggtacgag

SEQ ID NO: 119 - DNA, 25 bp, Artificial sequence
  synthetic sequence
caccggtgaaattctaaccttggcg

SEQ ID NO: 120 - DNA, 25 bp, Artificial sequence
  synthetic sequence
caccgcatcaaaaggaatgacttcc

SEQ ID NO: 121 - DNA, 25 bp, Artificial sequence
  synthetic sequence
caccgatcctggatgccaatcagct

SEQ ID NO: 122 - DNA, 25 bp, Artificial sequence
  synthetic sequence
caccgcgactgtggagaagagatcc

SEQ ID NO: 123 - DNA, 25 bp, Artificial sequence
  synthetic sequence
caccgcctcttgcttctccagatca